SEQUENCE LISTING (45 sequences)

SEQ ID NO: 1 - protein, 18 aa, Homo sapiens
SRSRYYRQRQRSRRRRRR

SEQ ID NO: 2 - protein, 26 aa, Homo sapiens
AKKAKSPKKAKAAKPKKAPKSPAKAK

SEQ ID NO: 3 - protein, 10 aa, Adenovirus
SGPSNTPPEI

SEQ ID NO: 4 - protein, 9 aa, Human papillomavirus
RAHYNIVTF

SEQ ID NO: 5 - protein, 10 aa, Human papillomavirus
TDLYCYEQLN

SEQ ID NO: 6 - protein, 10 aa, Human papillomavirus
AEPDRAHYNI

SEQ ID NO: 7 - protein, 19 aa, Human papillomavirus
KCDSTLRLCVQSTHVIRTL

SEQ ID NO: 8 - protein, 10 aa, Human papillomavirus
GTLGIVCPIC

SEQ ID NO: 9 - protein, 10 aa, Epstein-Barr Virus
DTPLIPLTIF

SEQ ID NO: 10 - protein, 15 aa, Epstein-Barr Virus
PRSPTVFYNIPPMPL

SEQ ID NO: 11 - protein, 9 aa, Epstein-Barr Virus
FLRGRAYGL

SEQ ID NO: 12 - protein, 15 aa, Epstein-Barr Virus
RGIKEHVIQNAFRKA

SEQ ID NO: 13 - protein, 10 aa, Epstein-Barr Virus
EENLLDFVRF

SEQ ID NO: 14 - protein, 9 aa, Epstein-Barr Virus
IVTDFSVIK

SEQ ID NO: 15 - protein, 9 aa, Homo sapiens
LLGRNSPEV

SEQ ID NO: 16 - protein, 13 aa, Murine sarcoma virus
KLVVVGARGVGKS

SEQ ID NO: 17 - protein, 12 aa, Homo sapiens
KLVVVGAVGVGK

SEQ ID NO: 18 - protein, 16 aa, Homo sapiens
DILDTAGLEEYSAMRD

SEQ ID NO: 19 - protein, 8 aa, Homo sapiens
GLEEYSAM

SEQ ID NO: 20 - protein, 10 aa, Homo sapiens
ELVSEFSRMA

SEQ ID NO: 21 - protein, 15 aa, Homo sapiens
HLDMLRHLYQGCQVV

SEQ ID NO: 22 - protein, 15 aa, Homo sapiens
SRLLGICLTSTVQLV

SEQ ID NO: 23 - protein, 9 aa, Homo sapiens
EADPTGHSY

SEQ ID NO: 24 - protein, 10 aa, Homo sapiens
LLDGTATLRL

SEQ ID NO: 25 - protein, 9 aa, Homo sapiens
YLEPGPVTA

SEQ ID NO: 26 - protein, 9 aa, Homo sapiens
MLLAVLYCL

SEQ ID NO: 27 - protein, 9 aa, Homo sapiens
YMNGTMSQV

SEQ ID NO: 28 - protein, 9 aa, Homo sapiens
YMNGTMSEV

SEQ ID NO: 29 - protein, 21 aa, Homo sapiens
AAGIGILTVILGVLLLIGCWY

SEQ ID NO: 30 - protein, 9 aa, Simian virus 40
TPPKKKRKV

SEQ ID NO: 31 - protein, 14 aa, Homo sapiens
KKSAKKTPKKAKKP

SEQ ID NO: 32 - protein, 26 aa, Homo sapiens
AKKAKSPKKAKAAKPKKAPKSPAKAK

SEQ ID NO: 33 - protein, 18 aa, Homo sapiens
SRSRYYRQRQRSRRRRRR

SEQ ID NO: 34 - protein, 255 aa, Artificial Sequence
  Description of Artificial SequenceHuman/murine chimeric single chain binding polypeptide (C6.5 sFv)
QVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLG

SEQ ID NO: 35 - DNA, 765 bp, Artificial Sequence
  Description of Artificial SequenceHuman/murine chimeric single chain binding polypeptide (C6.5 sFv)
caggtgcagctgttgcagtctggggcagagttgaaaaaacccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatggtcacaccaatcggcccgcaggggtccctgaccgattctctggctccaagtctggcacctcagcctccctggccatcagtgggttccggtccgaggatgaggctgattattactgtgcagcatgggatgacagcctgagtggttgggtgttcggcggagggaccaagctgaccgtcctaggt

SEQ ID NO: 36 - protein, 269 aa, Artificial Sequence
  Description of Artificial SequenceHuman/murine chimeric single chain binding polypeptide (C6ML3-9 sFv′)
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAAHHHHHHGGGGC

SEQ ID NO: 37 - DNA, 807 bp, Artificial Sequence
  Description of Artificial SequenceHuman/murine chimeric single chain binding polypeptide (C6ML3-9 sFv′)
caggtgcagctggtgcagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggaggaaccaagctgaccgtcctaggtgcggccgcacaccatcatcaccatcacggtggtggcggctgc

SEQ ID NO: 38 - protein, 282 aa, Artificial Sequence
  Description of Artificial SequenceHuman/murine chimeric single chain binding polypeptide (C6ML-3-9sFv′-L1-KDEL)
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAAHHHHHHGGGGCLESSSSGSEKDEL

SEQ ID NO: 39 - DNA, 846 bp, Artificial Sequence
  Description of Artificial SequenceHuman/murine chimeric single chain binding polypeptide (C6ML-3-9sFv′-L1-KDEL)
caggtgcagctggtgcagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggaggaaccaagctgaccgtcctaggtgcggccgcacaccatcatcaccatcacggtggtggcggctgcctcgagtcctctagctctggatccgaaaaagatgaactg

SEQ ID NO: 40 - protein, 287 aa, Artificial Sequence
  Description of Artificial SequenceHuman/murine chimeric single chain binding polypeptide (C6ML3-9sFv′-L2-KDEL)
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAAHHHHHHGGGGCLESSSSGSSSSGSEKDEL

SEQ ID NO: 41 - DNA, 861 bp, Artificial Sequence
  Description of Artificial SequenceHuman/murine chimeric single chain binding polypeptide (C6ML3-9sFv′-L2-KDEL)
caggtgcagctggtgcagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggaggaaccaagctgaccgtcctaggtgcggccgcacaccatcatcaccatcacggtggtggcggctgcctcgagtctagcagctccggttcctctagctctggatccgaaaaagatgaactg

SEQ ID NO: 42 - protein, 296 aa, Artificial Sequence
  Description of Artificial SequenceHuman/murine chimeric single chain binding polypeptide (C6ML3-9sFv′-L2-H14)
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAAHHHHHHGGGGCLESSSSGSSSSGSKKSAKKTPKKAKKP

SEQ ID NO: 43 - DNA, 888 bp, Artificial Sequence
  Description of Artificial SequenceHuman/murine chimeric single chain binding polypeptide (C6ML3-9sFv′-L2-H14)
caggtgcagctggtgcagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggaggaaccaagctgaccgtcctaggtgcggccgcacaccatcatcaccatcacggtggtggcggctgcctcgagtctagcagctccggttcctctagctctggatccaagaaaagcgcgaaaaagaccccgaagaaagcgaagaaaccg

SEQ ID NO: 44 - protein, 291 aa, Artificial Sequence
  Description of Artificial SequenceHuman/murine chimeric single chain binding polypeptide (C6ML3-9sFv′-L2-nls)
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAAHHHHHHGGGGCLESSSSGSSSSGSTPPKKKRKV

SEQ ID NO: 45 - DNA, 873 bp, Artificial Sequence
  Description of Artificial SequenceHuman/murine chimeric single chain binding polypeptide (C6ML3-9sFv′-L2-nls)
caggtgcagctggtgcagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggaggaaccaagctgaccgtcctaggtgcggccgcacaccatcatcaccatcacggtggtggcggctgcctcgagtctagcagctccggttcctctagctctggatccactccgccgaaaaagaaacgtaaagtg

(19) United States
(12) Patent Application Publication
(10) Pub. No.: US 2002/0132990 A1
(43) Pub. Date: Sep. 19, 2002

(54) Bioengineered vehicles for targeted nucleic acid delivery

(76) Inventors: James S. Huston, Chestnut Hill, MA (US); Pierre Wils, Paris (FR); Quan Zhu, Needham, MA (US); Oliver Laurent, Berkley, CA (US); Wayne A. Marasco, Oakland, CA (US); Daniel Scherman, Paris (FR)

Correspondence Address: Patrick J. Kelly, Synnestvedt & Lechner LLP, 2600 Aramark Tower, 1101 Market Street, Philadelphia, PA, 19107, US

(21) Appl. No.: 09/888,721

(22) Filed: Jun. 25, 2001

Related U.S. Application Data
(60) Provisional application No. 60/213,653, filed on Jun. 23, 2000.

(51) Int. Cl. A61K 48/00; C07K 16/46

(52) U.S. Cl. 530/391.1; 514/8

(57) ABSTRACT

There is disclosed a gene-delivery compound comprising: (A) a single-chain binding polypeptide having at least one effector segment which includes at least one cysteinyl residue; and (B) a nucleic acid-binding moiety which is coupled to the polypeptide via the cysteinyl residue. There is disclosed also a gene-delivery compound comprising: (A) a single-chain, binding polypeptide having at least one effector segment which includes at least one cysteinyl residue; (B) a lipid-associating moiety which is coupled to the polypeptide via the cysteinyl residue. Additionally disclosed are compositions comprising the above-mentioned compounds and a nucleic acid.

### CROSS-REFERENCE TO RELATED APPLICATIONS

This application is based on and claims priority to U.S. Provisional Application No. 60/213,653, filed Jun. 23, 2000.

### FIELD OF THE INVENTION

This invention is directed to targeted gene delivery compounds, methods for their production, and methods of their use. More particularly, the compounds of the invention are combinations of at least two molecules, one of which binds a nucleic acid and the other of which binds to a particular molecular marker on target cells. The compound delivers the nucleic acid to the target cell by binding the molecular marker and delivering the nucleic acid to the inside of the cell.

This invention relates more specifically to biosynthetic constructs of single-chain binding proteins, particularly single-chain Fv (sFv) species conjugated to nucleic acid-binding moieties or lipid-associating moieties.

### REPORTED DEVELOPMENTS

Various publications have described biosynthetic-binding polypeptides used for immunotargeting. Huston et al. (1988) describe the first biosynthetic single-chain Fv protein that was shown to be equivalent to the Fab fragment of the corresponding IgG, under the experimental conditions used. Huston and Oppermann in U.S. Pat. Nos. 5,091,513 and 5,132,405 describe single-chain Fv antibody fusion proteins which could be used alone or linked, via their amino or carboxy terminal fusion partners, to a bioactive amino acid sequence. Ladner et al., in U.S. Pat. No. 5,260,203, disclose a single-chain Fv binding protein having binding affinity for specific antigens and methods for producing genetic sequences coding for such peptides. Huston et al., in U.S. Pat. No. 5,753,204, disclose a formulation comprising a biosynthetic construct comprising disulfide-bonded single-chain Fv dimers. The formulations are said to have particular utility in in vivo imaging and drug targeting experiments. U.S. Pat. No. 5,877,305 to Huston et al. relates to single-chain Fv binding proteins capable of binding the c-erbB-2 (HER 2) or c-erbB-2-related tumor antigens.

A variety of publications have described the use of vectors comprising antibodies or single-chain binding polypeptides to deliver a compound to a given target in the body. Foster et al. describe an antibody complexed with a nucleic acid-binding moiety (Foster et al., Human Gene Therapy, 8:719-727 (1997)). Uherek et al. disclose a chimeric protein containing a Gal4 DNA-binding region fused to a single-chain Fv binding polypeptide (Uherek et al., J. Biol. Chem. 273:8835-8841 (1998)).

The use of lipidic vectors for the transfection of nucleic acid has been described in a variety of publications. Epand et al., in U.S. Pat. No. 5,283,185, disclose cationic lipidic vectors for use in the transfection of nucleic acids. Various publications have also described the use of lipidic vectors which additionally comprise targeting elements, including antibodies. Lee et al., in U.S. Pat. No. 5,908,777, disclose lipidic vectors which are useful for transfection of nucleic acid and which may contain ligands such as cell receptor-targeting ligands, fusogenic ligands, nucleus-targeting ligands, or a combination of such ligands. Huang et al., in U.S. Pat. No. 4,925,661, disclose liposomal vectors containing antibodies as targeting ligands for use in delivering cytotoxic reagents. Huang et al., in U.S. Pat. No. 4,957,735, disclose liposomal vectors containing antibodies as targeting ligands for use in delivering drugs, enzymes, hormones, DNA and other biomedically important substances. Huang et al., in U.S. Pat. No. 6,008,202, disclose cationic lipidic vectors containing antibodies as targeting ligands for use in the transfection of nucleic acids, polyanionic proteins, polysaccharides and other macromolecules which can be complexed directly with cationic lipids.

### SUMMARY OF THE INVENTION

In accordance with the present invention, there is provided a gene-delivery compound comprising: (A) a single-chain binding polypeptide having at least one effector segment which includes at least one cysteinyl residue; and (B) a nucleic acid-binding moiety which is coupled to said polypeptide by the cysteinyl residue.

In preferred form, the compound of the present invention includes a binding region which is effective in binding a surface marker of a mammalian cell and which comprises a single-chain Fv protein. Also in preferred form, the compound of the present invention includes an additional effector segment that, for example, binds reversibly with nucleic acids or facilitates endosomal escape or avoidance, or facilitates non-endosomal transport in a cell, or facilitates entry into the nucleus of a targeted cell. In another preferred embodiment, the compound of the present invention comprises also at least one spacer sequence, for example, a spacer sequence located between said effector segment containing said cysteinyl residue and an additional effector segment. In yet another preferred embodiment, the compound of the present invention further comprises a heterobifunctional crosslinking agent which couples said cysteinyl residue to said nucleic acid-binding moiety.

Another aspect of the present invention comprises a composition which includes the aforementioned compound of the present invention and a nucleic acid which is associated reversibly with the nucleic acid-binding moiety.

An additional aspect of the present invention is a gene delivery compound comprising: (A) a single-chain binding polypeptide having at least one effector segment which includes at least one cysteinyl residue; and (B) a lipid associating moiety which is coupled to said polypeptide by the cysteinyl residue.

In preferred form, the compound of the present invention having the lipid-associating moiety comprises an additional effector segment that is capable of associating with nucleic acid or facilitates endosomal escape or facilitates non-endosomal transport in the cell or facilitates entry into the nucleus of a cell. Also in preferred form, the present compound further comprises at least one spacer sequence located between said effector segment containing the cysteinyl residue and an additional effector segment.

In yet another aspect of the present invention, the invention provides a composition which includes the compound having the lipid-associating moiety and a liposome in association with the lipid-associating moiety. In preferred form, the composition comprises a nucleic acid in association with the liposome.

In preferred embodiments of the present invention, the single-chain binding polypeptide of each of the compounds of the present invention is effective in binding a surface marker of a mammalian cell, for example, a marker which is a tumor antigen.

The nucleic acid present in the compositions of the present invention preferably comprises DNA encoding a therapeutic gene, for example, lymphokines, tumor necrosis factors, intrabodies, tumor suppressor genes, p53, proapoptotic genes, suicide genes, prodrug converting genes, HSV-TK and anti-angiogenic genes.

### BRIEF DESCRIPTION OF THE DRAWINGS

FIG. 1 is a diagrammatic representation of a single-chain binding polypeptide of the present invention. Part (a) is the extended polypeptide format, and Part (b) is the folded protein format;

FIG. 2 is a diagrammatic representation of a single-chain binding polypeptide of the present invention illustrating the location of the complementarity determining regions, the polypeptide spacer regions, and the effector regions;

FIG. 3 is the amino acid sequence for C6.5 sFv;

FIG. 4 is the nucleotide sequence for C6.5 sFv;

FIG. 5 is the amino acid sequence for C6ML3-9 sFv′;

FIG. 6 is the nucleotide sequence for C6ML3-9 sFv′;

FIG. 7 is the amino acid sequence for C6ML3-9 sFv′-L1-KDEL;

FIG. 8 is the nucleotide sequence for C6ML3-9 sFv′-L1-KDEL;

FIG. 9 is the amino acid sequence for C6ML3-9 sFv′-L2-KDEL;

FIG. 10 is the nucleotide sequence for C6ML3-9 sFv′-L2-KDEL;

FIG. 11 is the amino acid sequence for C6ML3-9 sFv′-L2-H14;

FIG. 12 is the nucleotide sequence for C6ML3-9 sFv′-L2-H14;

FIG. 13 is the amino acid sequence for C6ML3-9 sFv′-L2-nls; nls is the SV40 large T antigen nuclear localization signal.

FIG. 14 is the nucleotide sequence for C6ML3-9 sFv′-L2-nls;

FIG. 15 shows that C6ML3-9 sFv′ and its conjugate to salmon protamine (SP) bind specifically to erbB-2 positive ovarian cancer cells;

FIG. 16 shows a FACS analysis of the erbB-2 binding activities of bacterially expressed C6ML3-9 sFv′ and its derivatives;

FIG. 17 is a gel shift analysis of C6.5 sFv′-SP-DNA and C6ML3-9 sFv′-SP-DNA complexes;

FIG. 18 shows a kinetic study of C6.5 sFv′-SP-DNA and C6ML3-9-SP-DNA complex formation;

FIG. 19 shows that a C6ML3-9 sFv-SP conjugate protein mediates specific luciferase gene delivery to erbB-2 positive cancer cells;

FIG. 20 illustrates chloroquine-dependence of C6ML3-9 sFv′-SP-mediated gene delivery;

FIG. 21 illustrates fluorescent microscopy of C6.5 sFv′-SP and C6ML3-9 sFv′-SP-mediated gene transfer of pGeneGrip Rhodamine/GFP plasmids with SK-OV-3 and MCF-7;

FIG. 22 illustrates the effect of chloroquine on 3T3-HER2 transfection mediated by C6ML3-9 sFv′-salmon protamine;

FIG. 23 illustrates the effect of chloroquine on 3T3-HER2 transfection mediated by C6ML3-9 sFv′-P1;

FIG. 24 illustrates the effect of chloroquine on 3T3-HER2 transfection mediated by C6ML3-9 sFv′-H1;

FIG. 25 illustrates the effect of C6ML3-9 sFv′-H1-pBks on 3T3-HER2 transfection mediated by C6ML3-9 sFv′-H1; and

FIG. 26 illustrates the effect of the DNA to C6ML3-9 sFv′-H1 ratio on 3T3-HER2 transfection efficiency.

### DETAILED DESCRIPTION OF THE INVENTION

The present invention is directed to gene delivery compounds which provide targeted non-viral delivery of genes to target cells. Such compounds comprise single-chain Fv proteins from antibodies, or analogues from the Ig superfamily, coupled to either a nucleic acid-binding moiety or a lipid-associating moiety.

The single-chain Fv combining site recognizes a given target antigen, such as a cell surface marker, and is fused to effector segments that provide further functional properties to the binding polypeptide. The binding proteins of the present invention preferably include at least one effector segment which contains at least one unpaired cysteinyl residue that may be used to form a linkage between the binding protein and a nucleic acid-binding moiety or a lipid-associating moiety. The binding protein may additionally include spacer segments which separate the binding regions in the binding protein and the effector regions from one another.

There is set forth hereafter a description of the compounds and compositions of the present invention and of each of the elements which comprise the compounds and compositions.

The Single-Chain Binding Polypeptide

The single-chain binding polypeptides of the present invention are typically based on the single-chain Fv antibody species known as “sFv” or “scFv” proteins. These sFv proteins have a binding site which exhibits the binding properties of an antibody combining site. The preparation of single-chain Fv protein has been previously described. See, for example, U.S. Pat. Nos. 5,091,513; 5,132,405; 5,258,498; 5,534,254; and 5,877,305 which are incorporated herein by reference.

A single-chain Fv binding protein includes at least two variable domains connected by a polypeptide linker or “spacer” which links the carboxy (C)-terminus of one domain to the amino (N)-terminus of the other domain. The amino acid sequences of each of the domains include a set of complementarity determining regions (CDRs) interposed between a set of framework regions (FRs). As used herein, a “set of CDRs” refers to 3 CDRs in each domain and a “set of FRs” refers to 4 FRs in each domain. The CDRs are held in an appropriate conformation by the FRs which are analogous to framework regions found in the Fv fragment of natural antibodies. When held in the proper three dimensional orientation by the FRs, the CDRs facilitate binding of the single-chain binding polypeptide to a desired antigen. Similar protein architecture is known in other members of the Ig super family, and they may be potentially used in a like manner.

The single-chain Fv binding proteins of the present invention define at least one complete combining site capable of binding to a desired antigen. One complete binding site includes a single continuous chain of amino acids having two polypeptide domains, that is, a variable heavy (V_{H}) and a variable light (V_{L}) domain, connected by an amino acid linker region. Binding polypeptides that include more than one complete binding site capable of binding an antigen, that is, two binding sites, comprise a single contiguous chain of amino acids having four polypeptide domains, each of which is covalently linked by an amino acid linker or spacer region, e.g., V_{H1}-linker-V_{L1}-spacer-V_{H2}-linker-V_{L2}. Binding polypeptides of the invention may include any number of complete binding sites (V_{Hn} linker V_{Ln})ₙ, where n>1, and thus may be a single contiguous chain of amino acids having n antigen-binding sites and nx2 polypeptide domains.

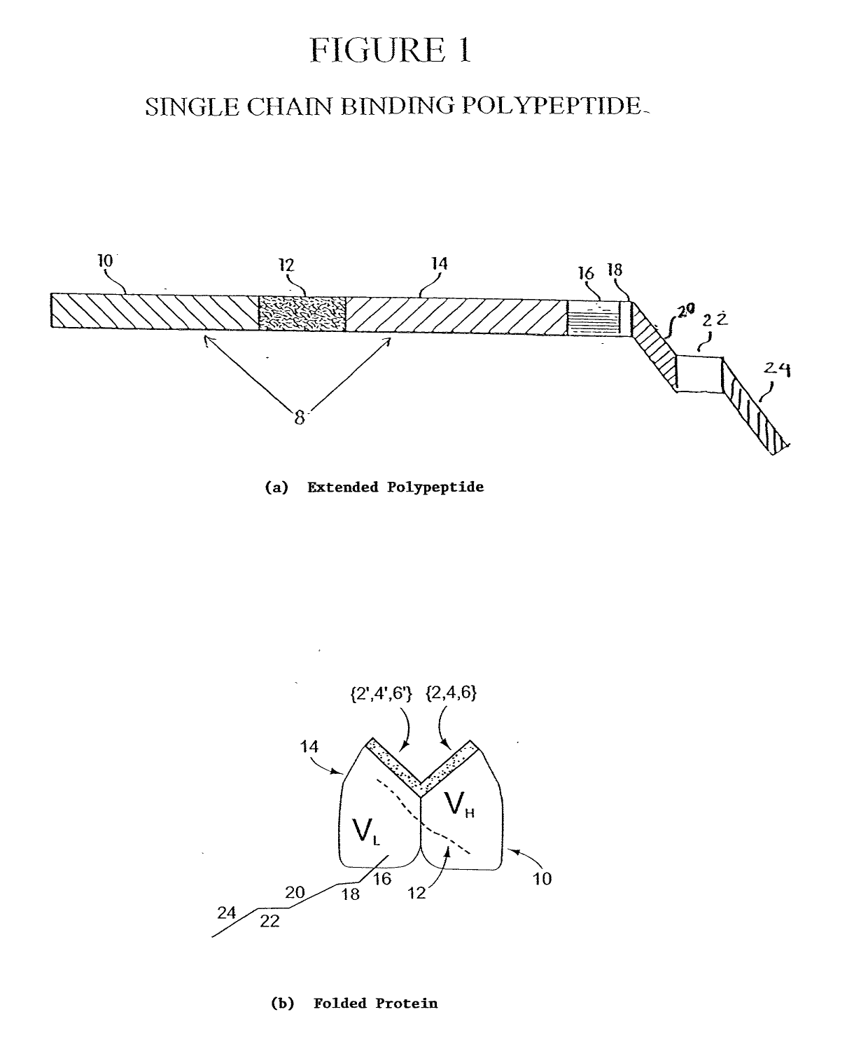

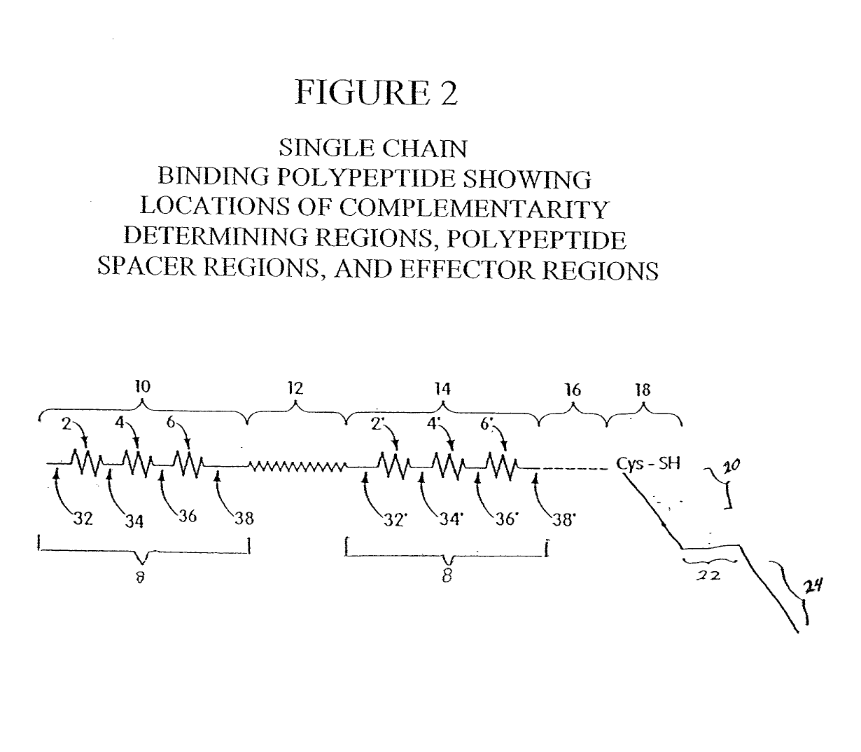

The single-chain Fv binding proteins of the invention can be further understood by referring to the accompanying FIGS. 1 and 2. FIG. 1 is a schematic representation of the single-chain Fv (sFv) polypeptide. FIG. 2 is a schematic representation of the sFv showing the locations of complementarity determining regions, polypeptide spacer regions, and effector regions. A native single-chain Fv (sFv), shown in FIGS. 1 and 2, comprises a heavy-chain variable region (V_{H}) 10 and a light-chain variable region, (V_{L}) 14. The V_{H} and V_{L} domains are compactly folded and are attached by polypeptide spacer 12. The binding domains defined by V_{H} and V_{L} include the CDRs 2, 4, 6 and 2′, 4′, 6′, respectively and FRs 32, 34, 36, 38 and 32′, 34′, 36′, 38′ respectively which, as shown in FIG. 2, together define an immunologically reactive binding site and Fv region 8. The sFv molecules contain also a C-terminal tail amino acid sequence 16 that will not self-associate with a polypeptide chain having a similar amino acid sequence under physiological conditions and which preferably contains an effector sequence, containing a cysteinyl residue 18 for the crosslinking of the single-chain binding polypeptide to a nucleic acid-binding moiety or lipid-associating moiety. This is followed by effector sequence 20. Spacer sequences (e.g., 22) can be used to separate the effector sequences from one another with additional effector sequences 24 providing additional functional abilities. The cys-containing segment and effector sequences may be ordered in any possible permutation, or additionally may be at the amino terminus of an sFv or within the linker connecting variable domains.

A variety of methods may be used. An sFv-phage antibody library is panned against a given target antigen thereby selecting sFv antibodies with appropriate specificities, which may be cloned and sequenced using conventional techniques. (See, for example, Marks, J. D., Antibody Engineering, 2d edition, C. Borrebaeck ed., pp. 53-88 (1995); Glover et al., DNA Cloning: A Practical Approach, Volumes I and II Oligonucleotide Synthesis, MRL Press, Ltd., Oxford, U.K. (1985)). The additional polypeptide segments may be designed empirically or be based on sequence analysis of appropriate protein sequences. Guidance on preparing single-chain binding polypeptides based on antibody sequences is provided in U.S. Pat. No. 5,132,405, which is incorporated herein by reference.

In certain situations, it may be desirable to perform mutagenesis of the antibody-binding regions, in particular, the complementarity determining CDRs of the single-chain binding polypeptide in order to increase the binding affinity of the single-chain binding polypeptide for its target antigen. Examples of suitable mutagenesis techniques to provide for enhanced binding are provided in Schier et al., J. Mol. Biol., 263, 551-567 (1996).

In one embodiment, the amino acid sequences constituting the FRs of the single-chain binding polypeptide are analogous to the FR sequences of a first preexisting antibody, for example, a human IgG. The amino acid sequences constituting the CDRs are analogous to the sequences from a second, different preexisting antibody, for example, the CDRs of a human IgG which recognizes a given antigen. Alternatively, the CDRs and FRs may be copied in their entirety from a single preexisting antibody from a cell line which may be unstable or difficult to culture, e.g., an sFv-producing cell line that is based upon a murine, mouse/human, or human monoclonal antibody-secreting cell line. The single-chain binding polypeptides may be prepared by recombinant DNA methods and the sequence encoding the binding polypeptides will be comprised of DNA made from ligation of chemically synthesized and recloned oligonucleotides or by ligation of fragments of DNA derived from the genome of a hybridoma, mature B cell clone, or a cDNA library derived from such natural sources. Because of structural considerations, an entire set of CDRs from an immunoglobulin may be used, but substitutions of particular residues may be desirable to improve biological activity, e.g., based on observations of conserved residues within the CDRs of immunoglobulin species which bind a given antigen. The binding polypeptides of the invention are able to refold into a 3-dimensional conformation selected to specifically exhibit affinity for a preselected antigen.

In embodiments intended for intravascular use in mammals, the FRs may include amino acid sequences that are similar or identical to at least a portion of the FR amino acids of antibodies native to that mammalian species. On the other hand, the amino acid sequences that include the CDRs may be analogous to a portion of the amino acid sequences from the hypervariable region (and certain flanking amino acids) of an antibody having a known affinity and specificity for a given antigen that is from, e.g., a mouse or rat, or a specific human antibody or immunoglobulin. Alternatively, the sFv binding region (or analogous Ig super family region) may be entirely of human composition for clinical use, or of some other mammalian source for other uses.

The present invention also provides for “multi-site targeting” utilizing single-chain binding polypeptides having the ability to bind to multiple, different surface markers on a target cell. Multi-site targeting with different epitopes or antigens enhances the selectivity of the binding polypeptide for its target cell, reducing the chance of binding to a non-target cell which has the same or similar surface markers as a target cell. Multi-site binding results in a more specific interaction with the target cell exhibiting the surface markers. A decreased binding affinity between a binding polypeptide and a surface marker reduces weak single-site binding and strongly favors selective binding of the binding polypeptide to a desired target cell. Accordingly, in this embodiment, a binding polypeptide may be used in which the binding affinity between the binding polypeptide and a surface marker (target antigen) is altered or decreased (i.e., reduced to lower than normal binding affinity). The decreased binding affinity can be accomplished by mutating the amino acid sequence of the binding regions of the binding polypeptide. In preferred embodiments, binding polypeptides having multiple surface marker-binding capacities have lower than normal binding affinity for the individual surface markers. To prepare these types of binding polypeptides, antibodies can be chosen with low binding constants (i.e., low affinity) for a given surface marker and the DNA cloned into the binding polypeptide. Alternatively, a lower binding constant can be achieved by using truncated, mutated, or otherwise altered peptide sequences. The multiple binding domains in these binding polypeptides are preferably spaced apart by amino acid spacer sequences to permit the binding polypeptide to bind to two or more surface markers on a surface cell. Preferably, the distance between the centers of two active binding sites would be about 60 to about 120 angstroms or greater for a less dense surface antigen.

Markers which may be bound by the single-chain binding polypeptide of the present invention include tumor antigens and tumor-associated antigens. In particular, such markers may be: erbB-2 (HER 2) (Foster and Kern, Human Gene Therapy, 8:719-727 (1997)), erbB-3 (HER 3) (Kraus et al., Proc. Natl. Acad. Sci. USA, 86(23):9193-7 (1989)), erbB-4 (HER 4) (Plowman et al., Proc. Natl. Acad. Sci. USA, 90(5):1746-50 (1993)), epidermal growth factor receptor, transferrin receptor (Thorstensen et al., Scand. J. Clin. Invest. Suppl., 215:113-120 (1993)), or Lewis^{Y} antigen (Ragupathi, G., Cancer Immunol. Immunother., 43(3): 152-7, Review (1996)).

Effector Sequences

An effector sequence is preferably included in the single-chain binding polypeptide and imparts additional functional properties to the binding polypeptide, for example, the ability to couple the binding polypeptide to another moiety, the ability to be taken into a cell, the ability to be taken into the nucleus of a cell, the ability to be expressed, and the ability to facilitate production or purification of the binding polypeptide.

It is believed that the effector sequence that will be used most widely in the practice of the present invention will be an effector sequence that facilitates coupling, cellular uptake and nuclear delivery of the nucleic acid. Both naturally-occurring and synthetic sequences may be utilized and the sequences may be prepared by subcloning or by oligonucleotide synthesis to prepare DNA sequences which encode the desired effector sequences.

Effector sequences that facilitate coupling may comprise a segment having amino acids which may couple with or are capable of being enzymatically modified so as to be able to couple the effector segment to a nucleic-acid binding moiety. For instance, glycosylation of an engineerred Asp-X-Ser sequence results in addition of a glycosyl residue suitable for chemical coupling. Preferably, effector sequences comprise a peptide sequence that includes a cysteinyl residue. In such embodiments the effector sequence is preferably a C-terminal sequence of at least about 5 amino acid residues including a cysteinyl residue. The single-chain binding polypeptide is conjugated directly or indirectly to a nucleic acid-binding moiety or a lipid-associating moiety via the thiol group on the cysteine residue, as described in more detail hereinbelow. The effector sequence is preferably fused to the C-terminus of the single-chain binding polypeptide via recombinant DNA techniques known in the art. The resulting fusion polypeptide is known as an sFv′. An example of fusing an effector sequence to a binding polypeptide is provided in Example 2. A preferred cysteine-containing effector sequence that facilitates crosslinking is Gly₄Cys.

Effector sequences may also include synthetic or natural fusogenic peptides such as GALA (Subbarao et al., Biochemistry, 2, 26(11), 2964-72 (1987)) or influenza haemagglutinin peptide HA (Wagner et al., Proc. Natl. Acad. Sci. USA, 89, 7934-38 (1992); Simoes et al., Gene Therapy, 5, 955-64 (1998)) which facilitate entry into target cells and escape from endosomes, facilitating delivery of genes to the cell nucleus for expression.

Effector sequences containing endoplasmic reticulum (ER) retention signals cause the complexed protein, in this case the gene delivery vehicle, to be targeted to the ER. The ER retention signals fused to the single-chain binding polypeptide, in particular the KDEL sequence, redirects the gene delivery vehicle to the ER through a KDEL-receptor-mediated retrieval mechanism (Pelham, Annu. Rev. Cell Biol., 5, 1-23 (1989); Zhu et al., J. Immunol. Methods, 231, 207-222 (1999)). The ER targeting/retention of the complexed protein/gene delivery vehicle may facilitate its endosomal escape and nuclear entry.

Effector sequences containing subcellular localization signals, such as nuclear localization signals (nls), cause a protein to be localized in the nucleus (Nigg, Nature, 386:779-787 (1997)). It is believed proteins recognize the nls, bind to it, and shuttle it and the complexed protein to the nucleus. A preferred nls is the SV-40 large T-antigen nuclear localization sequence TPPKKKRKV (Kalderon et al., Cell, 39, 499-509 (1984)). An example of a vehicle of the present invention including this sequence is provided in Example 2.

Spacer Sequences

Spacer sequences connect the C-terminus of one domain to the N-terminus of the next and provide flexibility for independent folding of the domains. The spacers preferably comprise hydrophilic amino acids which assume an unstructured configuration in physiological solutions and preferably are free of residues having large side groups which might interfere with proper folding of the V_{H}, V_{L}, or pendant chains. The spacers may be based on naturally-occurring sequences or may be synthesized. The spacers may be of any length that provides a sufficient distance between functional regions of the binding polypeptide such that the neighboring domains do not interfere with each other's functional activity. In preferred embodiments the spacer sequences are about 5 to about 20 amino acids, preferably about 15 amino acids. The spacer sequences may be subcloned from existing sequences or prepared via oligonucleotide synthesis and may be added to the binding polypeptide via standard molecular biological techniques. In preferred embodiments, the spacer sequences are prepared via oligonucleotide synthesis and incorporated into the single-chain binding polypeptide DNA via methods known in the art.

Examples of useful linker sequences include the amino acid sequence [(Gly)₄Ser]₃ and sequences comprising 2 or 3 repeats of [(Ser)₄Gly]₃. Preferred spacers include the same linker units for the region between the sFv binding domains of the binding polypeptide effector regions, as well as between the effector sequence(s), when multiple effector segments are present.

The Nucleic Acid-Binding Moiety

The nucleic acid-binding moiety may be any substance that binds to a nucleic acid. This binding may be covalent or non-covalent. The nucleic acid-binding moiety must be able to bind and retain the nucleic acid until the vehicle reaches and enters the target cell. The substance must not substantially damage or alter the nucleic acid due to its binding.

Preferably, the moiety is a polycation that binds electrostatically to negatively charged DNA or RNA. Examples of nucleic acid binding moieties include homologous organic polycations such as polylysine, polyarginine, polyornithine, and heterologous polycations having two ol more different positively charged amino acids, such as Arg-Lys mixed polymers. Non-peptidic synthetic polycations such as polyethyleneimine may also be used.

In preferred embodiments, nucleic acid-binding proteins of animal or vegetable origin are used, including histones, protamines, avidin, nucleolin, spermine or spermidines, high-mobility group (HMG) proteins, or analogues or fragments of these proteins, including peptides derived from these proteins.

Particularly preferred nucleic acid-binding proteins include salmon protamine, human protamine, a residue 11 to residue 28 subfragment of human protamine (SRSRYYRQRQRSRRRRRR), human histone H1 and a residue 166 to residue 192 subfragment of human histone H1 (AKKAKSPKKAKAAKPKKAPKSPAKAK).

The size of the nucleic acid binding moiety and its nucleic acid will be determined by the intended clinical use for the vehicle, in particular, on the ability of the nucleic acid to be taken up by its target cell. Preferably, the nucleic acid and the nucleic acid-binding moiety are compacted to a size which is sufficiently small for receptor mediated endocytosis, passive internalization, receptor-mediated membrane permeabilization, or other cell uptake mechanisms. In preferred embodiments, the target-binding moiety of the compacted nucleic acid and the nucleic acid-binding moiety is less than 1000 nm, and more preferably less than about 250 nm.

Lipid-Associating Moiety

In gene-delivery vehicles comprising a single-chain binding polypeptide crosslinked to a lipid-associating moiety, the lipid-associating moiety comprises a molecule capable of inserting into lipid-containing compositions such as micelles or the lipid bilayer of a liposome. The lipid-associating moiety may be any molecule sufficiently hydrophobic and sterically able to associate with and retain a lipid or liposome and facilitate delivery of the lipid or liposome to the inside of a target cell once the cell has been bound via the activity of the single-chain binding polypeptide. The lipid-associated moiety may be any molecule able to associate with lipids, micelles or liposomes, and remain associated with them. The lipid-associating moiety may be linear, branched, cyclic, poly-cyclic, saturated, or unsaturated and preferably includes a hydrophilic polymer to increase the distance between the lipid or liposome and the single-chain binding protein. The lipid-associating moiety may include a thiol reactive group, such as maleimide, alkyl and aryl halides, pyridyl disulfides, and α-halo-acyls to facilitate crosslinking with a cysteine residue on the single-chain binding polypeptide.

Examples of preferred lipid-associating moieties include maleimide-polyethylene glycol-dioctadecyl acetamide (Maleimide-PEG-(C18)₂) and maleimide-polyethyleneglycol-1,2-distearoyl-sn-glycero-3-phosphatidyl ethanolamine (maleimide-PEG-DSPE). More generally, the moiety could be any maleimide-activated phospholipid or PEG-bearing phospholipid.

A particularly preferred lipid-associating moiety is ((2-amino-PEG-ethylcarbamoyl)-methoxy)-N,N-dioctadecyl-acetamide. The two dioctadecyl chains form the hydrophobic portion of the amphipathic molecule, while the polyethylene glycol (“PEG”) forms the hydrophilic portion. In a preferred embodiment, the PEG has 65 to 85 oxyethyl units.

The Liposomes and Lipids

The nucleic acid may be encapsulated within a liposome or associated with a micelle. Liposomes or micelles are targeted to cells by surface bound sFv. For both liposomes and micelles, the transgene is incorporated into the target cells either by fusion of the carrier with the plasma membrane, or by endocytosis of the carrier.

Liposomes are lipid bilayer membranes containing an entrapped aqueous volume. Liposomes may be unilamellar vesicles (possessing a single membrane bilayer) or multilameller vesicles (onion-like structures characterized by multiple membrane bilayers, each separated from the next by an aqueous layer). These liposomes are preferably comprised of amphiphilic molecules such as amphiphilic lipids with or without a neutral lipid. Liposomes may be composed of phospholipids, sphingolipids, cholesterol, or a combination thereof. For the purposes of the present invention, the liposomes are preferably composed of cationic lipids, such as dioleoyltrimethylammoniumpropane (DOTAP), dimethyl-dioctacecylammonium bromide (DDAB), DC-chol, DOSPRA, DPPS, DPPES, DOGS and other cationic lipids such as those described in WO98/54130 and WO 97/18185. In addition to cationic lipids, liposomes preferably contain also “helper lipids” which promote the formation of liposomes, promote fusion with the cellular membranes (including endosomal membrane), promote endosomal escape (including by other means than membrane fusion), enhance the gene transfer efficacy, reduce interaction with serum, change the surface charge of the liposome, change the size of the liposome, and stabilize the liposome, such as dioleoylphosphatidyl-ethanolamine (DOPE) and cholesterol. (See Gao and Huang, Gene Therapy 2:710-722 (1995).)

Methods for preparing liposomes are well known in the art and include extrusion, reverse phase evaporation, detergent-dialysis processes, sonication, and microfluidization. The “reverse phase evaporation” (REV) process of Papahadjopoulos (U.S. Pat. No. 4,235,871, issued Nov. 25, 1980) forms oligolamellar lipid vesicles wherein the aqueous material to be encapsulated is added to lipids in an organic solvent, forming a water-in-oil type emulsion. The organic solvent is removed, forming a gel. The gel is dispersed in aqueous medium converting it to a suspension. The detergent-dialysis process (Enoch et al., 1979, Proc. Natl. Acad. Sci., 76:145) involves mixing a lipid with a detergent such as deoxycholate in aqueous solution, sonicating, and the removal of the detergent by gel filtration. A further technique is the ethanol infusion technique of Batzri et al. (1973, Biochim. Biophys. Acta., 298:1015), for forming small unilamellar vesicles, whereby an ethanol solution of lipid is injected into the desired aqueous phase, forming liposomes of about 30 nm to about 2 μm in diameter. The residual ethanol may then be removed by rotoevaporation. Unilamellar vesicles may also be produced using an extrusion apparatus by a method described in Cullis et al., PCT Application No. WO 86/00238, Jan. 16, 1986, entitled “Extrusion Technique for Producing Unilamellar Vesicles” incorporated herein by reference.

Another type of liposome which may be used in the practice of the present invention is a stealth liposome (Lasic, D. and Martin, F., eds. (1995) Stealth Liposomes, CRC Press). Stealth liposomes are less likely to be destroyed by the body's immune system due to the presence of a layer, preferably a hydrophillic layer, on the surface of the liposome which physically blocks interaction with other surfaces. One such example of a stealth liposome involves the attachment of polyethylene glycol to the surface of the liposome using a lipid anchor.

Another class of liposomes that may be used in the present invention are those characterized as having substantially equal lamellar solute distribution. This class of liposomes is designated as stable plurilamellar vesicles (SPLV) as described in U.S. Pat. No. 4,522,803 to Lenk, et al., monophasic vesicles as described in U.S. Pat. No. 4,588,578 to Fountain, et al., and frozen and thawed multilamellar vesicles (FATMLV) which are exposed to at least one freeze and thaw cycle; this procedure is described in Bally et al., PCT Publication No 87/00043, Jan. 15, 1987, entitled “Multilamellar Liposomes Having Improved Trapping Efficiencies”. The relevant portions of the aforementioned publications are incorporated herein by reference.

Cationic lipids may also be used to form micelles (Pitard et al., PNAS 94:14412-14417 (1997)). Micelles are non-vesicular colloids of amphiphilic molecules having a hydrophobic “tail” region and a hydrophilic “head” region. The structure of the micelle is such that the hydrophobic (nonpolar) “tails” of the amphiphilic molecules orient toward the center of the micelle while the hydrophilic “heads” orient towards the aqueous phase.

In vehicles utilizing liposomes, the nucleic acid may be encapsulated in the liposomes. In both cationic micelle and cationic liposome formations, the nucleic acid is associated through charge interactions with cationic lipids or cationic liposomes to form “cationic lipid/nucleic acid complexes” or “lipoplexes”. Felgner et al., Human Gene Therapy 8:511-512 (1997). The structures of these complexes have been described in Radler et al., Science 275:810-814 (1997), Pitard et al., PNAS 94:14412-14417 (1997), and Koltover et al., Science 281:78-81 (1998).

The nucleic acid to be delivered is preferably first condensed with cationic peptides or cationic polymers and mixed with lipids or liposomes. Cationic lipid/DNA complexes are preferably also modified or coated with PEG or other inert hydrophilic polymers to give stealth liposomes or sterically stabilized liposomes non-immunogenic properties. (Lasic, Trends Biotech. 16:307-321 (1998).)

In the present invention, single-chain binding polypeptides are used as fusion proteins with binding specificity to target the lipid/nucleic acid complex to specific cells. Single-chain binding polypeptides may be associated with the lipid/nucleic acid complex by various methods. Single-chain binding polypeptide-lipid conjugates can be first associated with cationic lipids then mixed with nucleic acid, or lipid/nucleic acid complexes can be formed first, then single-chain binding polypeptide-lipid conjugates incorporated in these complexes.

Crosslinking of the Single-Chain Binding Polypeptide to the Nucleic Acid-Binding Moiety or Lipid-Associating Moiety

The single-chain binding polypeptide may be coupled with either the nucleic acid-binding moiety or the lipid-associating moiety by any coupling method recognized in the art as capable of coupling such moieties. Preferably, the two moieties are covalently coupled.

It is preferable that at least one moiety to be coupled contains a thiol group. In the most preferred embodiments, the single-chain binding polypeptide includes an effector sequence which includes a cysteine residue. In embodiments in which the single-chain Fv antibody moiety contains a reactive thiol group, the moiety to be coupled with the single-chain Fv antibody preferably contains, or is complexed with, a thiol-reactive group. Essentially any thiol-reactive group known in the art may be used. Examples of such groups include but are not limited to: maleimide; alkyl halides; aryl halides; pyridyl disulfides; and α-halo-acyls.

In preferred embodiments, crosslinking reagents are utilized to couple the single-chain binding polypeptide with either the nucleic acid-binding moiety or the lipid-associating moiety. Essentially any crosslinking reagent recognized in the art as capable of crosslinking proteins to other proteins may be employed.

Crosslinking reagents function in various ways. Some crosslinking reagents become incorporated into the final product while some do not. Additionally, some crosslinking reagents are homofunctional in that they react only with like-functional groups while others are heterofunctional in that they react with different functional groups. Bifunctional crosslinking reagents are reagents that react with two functional groups. Bifunctional crosslinking reagents may be either heterofunctional (“heterobifunctional”) or homofunctional (“homobifunctional”).

The crosslinking reagents that will be used most widely in the practice of the present invention will be the heterobifunctional crosslinking reagents. Heterobifunctional crosslinking reagents which react with thiol groups and amine groups are particularly preferred. An effective amount of the crosslinking reagent is used to form the crosslink. The amount may be readily determined by those of ordinary skill in the art without undue experimentation. Preferably, when coupling the heterobifunctional crosslinker to SP, the amount of crosslinking reagent is sufficient to stoichiometrically label the α-amino group of SP. For optional yields of the sFv′-SP conjugate, it is recommended that an excess of modified SP be mixed with the sFv′ having at least one available SH group. A variety of crosslinking agents are known in the art. Examples of useful crosslinking agents are described in Hermanson, G. T., “Bioconjugate Techniques”, Academic Press, 1996. Examples of such reagents include but are not limited to: SPDP (N-succinimidyl 3(2-pyridyldithio)propionate); LC-SPDP; sulfo-LC-SPDP; MBS (maleimidobenzoyl-N-hydroxysuccinimide ester); sulfo-MBS; SIAB (N-succinimidyl(4-iodoacetyl)-aminobenzoate); sulfo-SIAB; SMCC (succinimidyl 4-[N-maleimidomethyl]cyclohexane-1-carboxylate); and sulfo-SMCC.

In embodiments of the present invention in which a crosslink between amine and thiol groups is desired, succinimidyl trans-4(maleimidylmethyl)-cyclohexane-1-carboxylate (SMCC) and its water-soluble variant Sulfo-SMCC are the preferred heterobifunctional crosslinking reagents. In preferred embodiments, the nucleic acid-binding moiety is reacted with Sulfo-SMCC (Pierce Cat. No. 22322) and the resulting conjugate contains a thiol-reactive maleimide. The maleimide reacts with the thiol group of the cysteinyl-residue complexed with the single-chain binding polypeptide. This results in crosslinking of the nucleic acid binding moiety with the single-chain binding polypeptide.

An example of utilizing SMCC to crosslink a single-chain binding polypeptide with salmon protamine conjugate is described in Example 6.

The Nucleic Acid Being Delivered

In the compositions of the present invention, the nucleic acid can be either a deoxyribonucleic acid or a ribonucleic acid. The sequences in question can be of natural or artificial origin, and in particular genomic DNA, cDNA, mRNA, tRNA, rRNA, hybrid sequences or synthetic or semi-synthetic sequences. In addition, the nucleic acid can be variable in size, ranging from small plasmids or oligonucleotides to chromosome. These nucleic acids may be from a variety of sources, including human, animal, vegetable, bacterial, and viral origin. They may be obtained by any technique known to a person skilled in the art, in particular by the screening of libraries, by chemical synthesis or alternatively by mixed methods including the chemical or enzymatic modification of sequences obtained by the screening of libraries. They can, moreover, be incorporated into vectors, such as plasmid vectors.

The deoxyribonucleic acids, may be single- or double-stranded. These deoxyribonucleic acids can carry therapeutic genes, sequences regulating transcription or replication, antisense sequences, regions for binding to other cell components, and the like.

For the purposes of the invention, the therapeutic gene may code for a proteinaceous product having a therapeutic effect. The proteinaceous product thus encoded can be a protein, a peptide, and the like. This proteinaceous product can be homologous with respect to the target cell (that is to say a product which is normally expressed in the target cell when the latter is not suffering from any pathology). In this case, the expression of a protein makes it possible, for example, to remedy an insufficient expression in the cell or the expression of a protein which is inactive or weakly active on account of a genetic abnormality, or alternatively to overexpress the said protein. The therapeutic gene may also code for a mutant of a cell protein, having enhanced stability, modified activity, and the like. The proteinaceous product may also be heterologous with respect to the target cell. In this case, an expressed protein may, for example, supplement or supply an activity which is deficient in the cell, enabling it to combat a pathology, or stimulate an immune response. The therapeutic gene may also code for a protein secreted into the body.

Therapeutic genes useful in the practice of the present invention include enzymes; blood derivatives; hormones; lymphokines, namely interleukins, interferons, tumor necrosis factor, and the like (FR 92/03120); growth factors; neurotransmitters or their precursors or synthetic enzymes; trophic factors, namely BDNF, CNTF, NGF, IGF, GMF, αFGF, βFGF, NT3, NT5, HARP/pleiotrophin, and the like; apolipoproteins, namely ApoAI, ApoAIV, ApoE, and the like (FR 93/05125); dystrophin or a minidystrophin (FR 91/11947); the CFTR protein associated with cystic fibrosis; intrabodies; tumor-suppressing genes, namely p53, Rb, Rap1A, DCC, k-rev, and the like (FR 93/04745); genes coding for factors involved in coagulation, namely factors VII, VIII, IX; genes participating in DNA repair; suicide genes (genes whose products cause the death of a cell; e.g., thymidine kinase (HS-TK), cytosine deaminase), and the like; pro-apoptic genes; prodrug converting genes (genes coding for enzymes who convert prodrugs to drugs); and anti-angiogenic genes, or alternatively, genes such as VEGF that promote angiogenesis.

In one embodiment, the nucleic acid can encode one or more genes encoding intrabody proteins. Intrabodies are described in U.S. Pat. No. 6,004,940. Delivery of the nucleic acid to a target cell provides for intracellular expression of the intrabody which is capable of intracellular binding to a specific target antigen. As used herein, the term “intrabody” refers to at least that portion of an immunoglobulin capable of selectively binding to a target such as a protein. Almost any molecule can serve as the target antigen for the intrabody, including intermediate metabolites, sugars, lipids, and hormones as well as macromolecules such as complex carbohydrates, phospholipids, nucleic acids such as RNA and DNA, and proteins. The preferred target molecules are proteins on the cell surface or proteins involved in intracellular signaling or metabolism. For example, the target may be p53 or the extracellular domain of erbB-2.

The therapeutic genes of the present invention can also be an antisense gene or sequence, whose expression in a target cell enables the expression of genes or the transcription of cellular mRNAs to be controlled. Such sequences can, for example, be transcribed in the target cell into RNAs complementary to cellular mRNAs and can thus block their translation into protein, according to the technique described in Patent EP 140,308. Other possible sequences include synthetic oligonucleotides, optionally modified (EP 92,574). Antisense sequences also comprise sequences coding for ribozymes, which are capable of selectively destroying target RNAs (EP 321,201).

As stated above, the nucleic acid can also contain one or more genes coding for an antigenic peptide capable of generating an immune response in man or animals. In this particular embodiment, the invention hence makes possible the production either of vaccines or of immunotherapeutic treatments applied to man or animals, in particular against microorganisms, viruses or cancers. Such peptides include, in particular, antigenic peptides specific to the Epstein Barr virus, the HIV virus, the hepatitis B (EP 185,573) or the pseudorabies virus, or alternatively tumor-specific peptides (EP 259,212).

Preferably, the nucleic acid also comprises sequences permitting the expression of the therapeutic gene in the desired cell or organ. These sequences can be the ones which are naturally responsible for expression of the gene in question when these sequences are capable of functioning in the infected cell. They can also be sequences of different origin (responsible for the expression of other proteins, or even synthetic sequences). In particular, they can be promoter sequences of eukaryotic or viral genes. For example, they can be promoter sequences originating from the genome of the cell which is to be genetically modified. Similarly, they can be promoter sequences originating from the genome of a virus. In this connection, the promoters of the E1A, MLP, CMV, RSV and like genes may be utilized. In addition, these expression sequences may be modified by the addition of activation or regulatory sequences or sequences permitting tissue-specific expression or inducible expression.

Moreover, the nucleic acid can also contain, especially upstream of the therapeutic gene, a signal sequence directing the therapeutic product synthesized into the pathways of secretion of the target cell. This signal sequence can be the natural signal sequence of the therapeutic product, but it can also be any other functional signal sequence, or an artificial signal sequence.

The non-viral gene delivery vehicle of choice, complexed with nucleic acid enters the target cells in amounts effective to achieve the desired therapeutic effect.

The Target Cell

The target cells may be located in a patient's nervous system, circulatory system, digestive system, respiratory system, reproductive system, endocrine system, skin, muscles, or connective tissue. In veterinary applications, similar target cells would be applicable.

The target cells of the present invention include any mammalian host cell. In particular, target cells can be tumor cells, virus-infected cells, bacteria-infected cells, or cells causing genetically based disease. The target cells have surface markers which are inherently present or which are present due to a disease condition. These surface markers may include specific receptors, or selective antigens, such as tumor-associated antigens. The type and number of surface markers of a cell provide a unique profile to that cell, distinguishing a given cell from other cells present in the host.

In preferred embodiments, the target cells are cancer cells derived from any organ or tissue in a patient.

The vehicles of the present invention are designed to deliver a nucleic acid to a target cell based on antigenic markers located on the target cell. Such markers may include erbB-2 (Foster and Kern, Human Gene Therapy, 8:719-727 (1997)), erbB-3 (Kraus et al., supra), erbB-4 (Plowman et al., supra), epidermal growth factor Receptor, transferrin receptor (Thorstensen et al., supra), Lewis antigen (Ragupathi, supra), and prostate specific membrane antigen (PSMA). Such markers may also include the following markers (as described in Kawakami and Rosenberg, Immunologic Research, 164/4:313-339 (1997)): K-ras; p53; Mage 1; Mage 3; gp 100; tyrosinase; Mart-1/Melan A; carcinoembryonic antigen (CEA); and prostate specific antigen (PSA). Various other tumor associated antigens may also be used, including, for example, the antigens identified in Storkus, W. and Lotze, M., Biologic Therapy of Cancer: Principles and Practice, Second Edition, Section 3.2, “Tumor Antigens Recognized by Immune Cells,” pp. 64-77, J. B. Lippincott Co. publishers (1995). A list of tumor-associated antigens which may be targeted by the single-chain binding proteins of the present invention are presented below in Table 1.

TABLE 1
Tumor-Associated Antigens and Peptide Epitopes

| Source | TAA | Amino Acid Sequence |
|---|---|---|
| Adenovirus | E1A | p234-243; SGPSNTPPEI |
| HPV-16 | E6/E7 | multiple putative epitopes |
| | E7 | p49-57; RAHYNIVTF |
| | E7 | p20-29; TDLYCYEQLN |
| | E7 | p45-54; AEPDRAHYNI |
| | E7 | p60-79; KCDSTLRLCVQSTHVIRTL |
| | E7 | p85-94; GTLGIVCPIC |
| EBV | EBNA-2 | p67-76; DTPLIPLTIF |
| | EBNA-2 | p276-290; PRSPTVFYNIPPMPL |
| | EBNA-3A | p33O-338; FLRGRAYGL |
| | EBNA-3C | p332-346; RGIKEHVIQNAFRKA |
| | EBNA-3C | p290-299; EENLLDFVRF |
| | EBNA-4/6 | p416-424; IVTDFSVIK |
| p53 | p53 | p264-272; LLGRNSPEV |
| p21^{ras} | ras | p5-17; KLVVVGARGVGKS |
| | ras | p5-16; KLVVVGAVGVGK |
| | ras | p54-69; DILDTAGLEEYSAMRD |
| | ras | p60-67; GLEEYSAM |
| HER2/neu | neu | p971-980; ELVSEFSRMA |
| | neu | p42-56; HLDMLRHLYQGCQVV |
| | neu | p783-797; SRLLGICLTSTVQLV |
| Human | MAGE1 | p161-169; EADPTGHSY |
| Melanoma | gp100 | p457-466; LLDGTATLRL |
| | gp100 | p280-288; YLEPGPVTA |
| | Tyrosinase | p1-9; MLLAVLYCL |
| | Tyrosinase | p368-376; YMNGTMSQV |
| | Tyrosinase | p368-376; YMNGTMSEV |
| | MART-1/Aa | p27-47; AAGIGILTVILGVLLLIGCWY |

Pharmaceutical Compositions and Methods

The compositions of the present invention may further comprise a carrier which is pharmaceutically acceptable for administration to an animal subject. Pharmaceutically acceptable carriers include solvents (e.g., phosphate-buffered saline), dispersion media, antibacterial agents, antifungal agents, and the like which are compatible with the maintenance of the proper conformation of the single-chain binding polypeptides and their use as non-viral gene delivery vehicles.

The compositions of the present invention may also further comprise supplementary active ingredients. Nuclease inhibitors and the like may be incorporated to protect the nucleic acid of the composition from degradation. MgCl and the like may be used to decrease the size of the DNA complex. Sucrose, dextrose, glycerol, and the like may be used to increase the stability of the DNA complex. Lysosomotropic agents such as chloroquine, monensine, and the like may be used to improve efficiency of the delivery of the nucleic acid.

The pharmaceutical compositions are preferably sterile. Sterilization may be achieved by any method known in the art, including filtration of the solution through a sterile filter and/or lyophilization followed by sterilization with a gamma ray source.

Administration of the composition of the present invention may be by any suitable method known in the art. Examples of such methods include, but are not limited to, intravascular and subcutaneous injection, topical application, and oral ingestion. The dosage may be determined by systematic testing of alternative doses until a suitable dosage level is identified. If a trial dose is too low to be effective, the dosage level may be increased. If a trial dose is so high as to be toxic, the dosage level may be decreased. Clinically, dosing schedules may be determined by using a dose escalation protocol with patients, thereby identifying the optimal dosing regime.

### EXAMPLES

### Example 1

Preparation of Single-Chain Binding Polypeptide C6ML3-9 sFv′

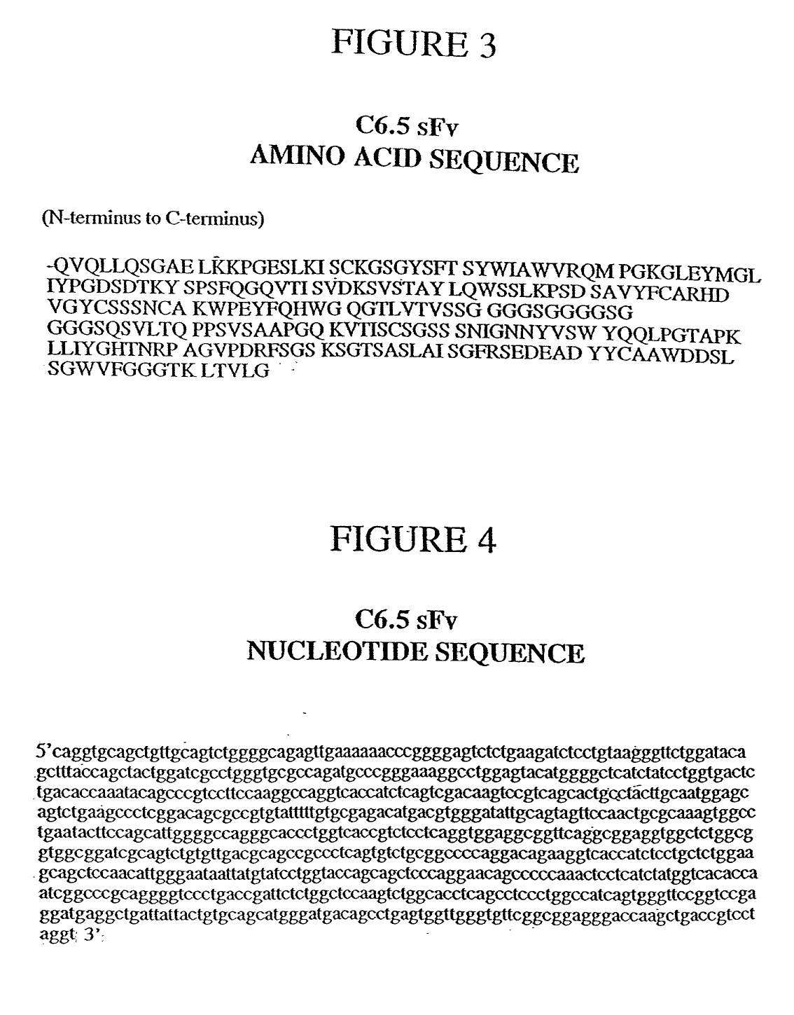

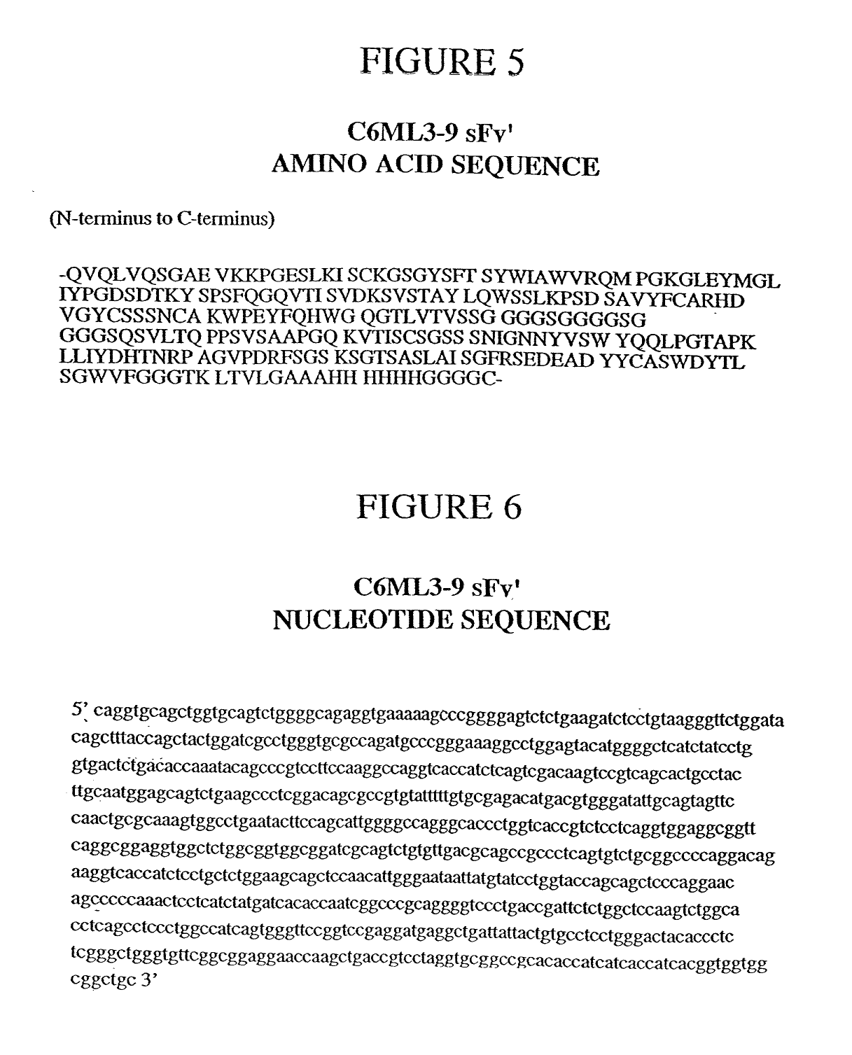

The single-chain binding polypeptides used in the following examples are based on two anti-c-erbB-2 single-chain sFvs. The C6.5 sFv was the first anti-erbB-2 described by Schier et al., Immunotechnology, Vol. 1, 73-81 (1995); a second analogue of this sFv, C6ML3-9 sFv, was described by Schier et al., J. Mol. Biol., Vol. 263, 551-567 (1996). C6ML3-9 sFv was prepared by modifying the complementarity determining regions (CDRs) of C6.5. The sequences of C6.5 and C6ML3-9 are presented in FIGS. 3 and 5. These sequences can be synthesized and cloned into appropriate vectors using standard molecular biological methods.

The following is a description for the construction of a single-chain binding protein based on C6ML3-9 sFv but this method may be used to convert C6.5 or any other suitable single-chain sFv into a single-chain binding protein suitable for use in the present invention. To convert C6ML3-9 sFv into C6ML3-9 sFv′, an oligonucleotide encoding the amino acid sequence His₆Gly₄Cys followed by a stop codon was fused in frame at the C-terminus of C6ML3-9 sFv using a NotI site.

The following is an example for the construction of C6ML3-9 sFv′.

The NcoI/NotI DNA fragment encoding C6ML3-9 sFv was excised out of a plasmid vector containing the sequence and inserted into the NcoI/NotI sites of a modified pET22-b(+) from Novagen. The pET22-b was modified by insertion of an oligonucleotide encoding the amino acid sequence His₆Gly₄Cys between the NotI and XhoI sites of the plasmids. The finished construct was named pETC6ML3-9 sFv′.

The NcoI/XhoI (blunt) DNA fragment encoding C6ML3-9 sFv′ was then excised out of pETC6ML3-9 sFv′ plasmid and inserted into the NcoI/EcoRI (blunt) sites of a pUC119 related vector (Schier et al., Immunotechnology, 1:73-81 (1995); Griffiths et al., EMBO, 13: 3245-3260 (1994)). The final construct is named C6ML3-9 sFv′ and used for production of C6ML3-9 sFv′ protein in TG1 bacterial cells. TGI bacterial cells can be obtained from Stratagene, Cat. #200123.

### Example 2

Genetic Construction and Protein Expression of C6ML3-9 sFv′ Fused with Different Effector Sequences

The following C6ML3-9 sFv′ derivatives were prepared in which the specific effector sequences were fused to the C-terminus of C6ML3-9 sFv′ in order to increase gene delivery due to endosomal escape and nuclear targeting. The vectors had the following insert:

Pel B-Sfi I-Nco I-sFv-Not I-His6-Gly4Cys-Xho I-Spacer (L1 or L2)-BamH I-effector sequence-stop-EcoR I,

The spacer L1=Ser₄Gly and the spacer L2=2×(Ser₄Gly).

Pel B is a secretion signal which directs the sFv′ into the periplasm of bacterial cells. The spacer L1 or L2 serves as a linker between sFv′ and the effector sequence, which makes the effector sequence available after the sFv′ is coupled to a nucleic acid binding moiety, in particular salmon protamine, or lipid-associating moiety. The effector sequences include:

(1) SEKDEL, an ER retention signal (Monro, S. and Pelham, H. R. B., Cell, 48:899-907, 1987), which had shown ER association in the absence of a typical leader sequence;

(2) the SV40 large T-antigen nuclear localization signal: TPPKKKRKV (Kalderon et al., Cell, 39:499-509 (1984)); and

(3) the amino acids 147-160 of human histone H1: KKSAKKTPKKAKKP; the C6ML3-9 sFv′ conjugated to a related histone peptide was shown previously to mediate low levels of luciferase gene transfer without chloroquine. Chloroquine tends to accumulate into the acidic compartments of the endocytic pathway. It increases their pH, induces their swelling and eventually their leakage. This may reduce lysosomal degradation and facilitate endosomal escape.

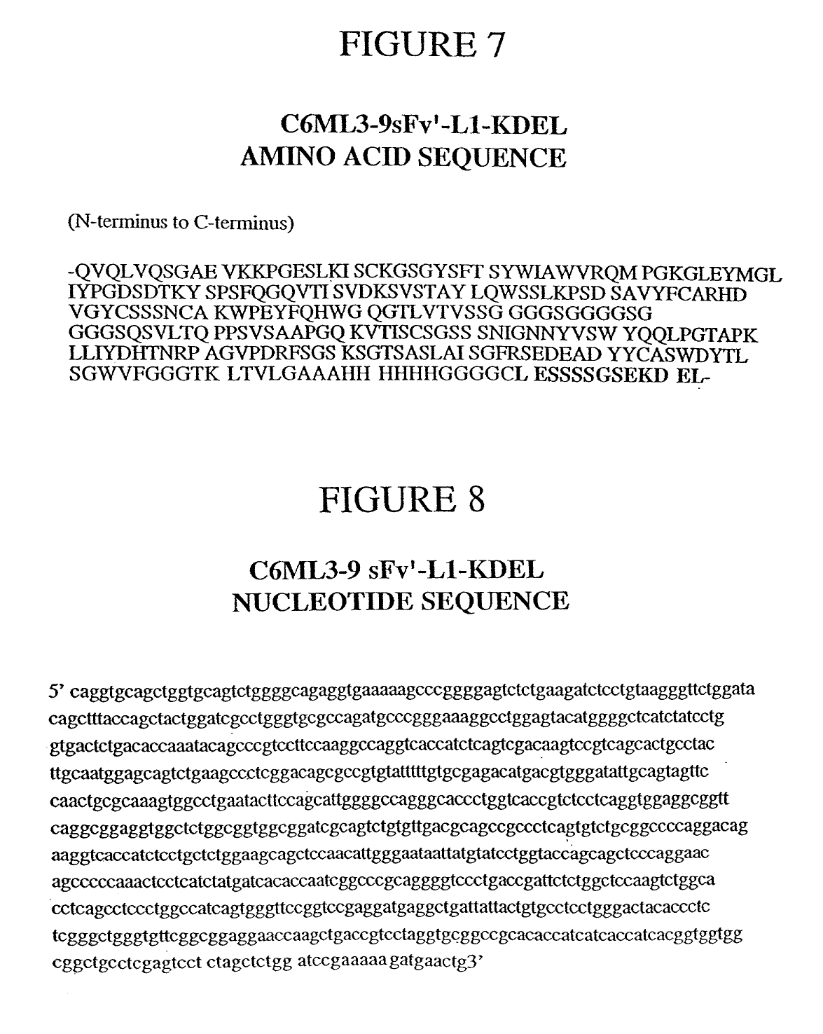

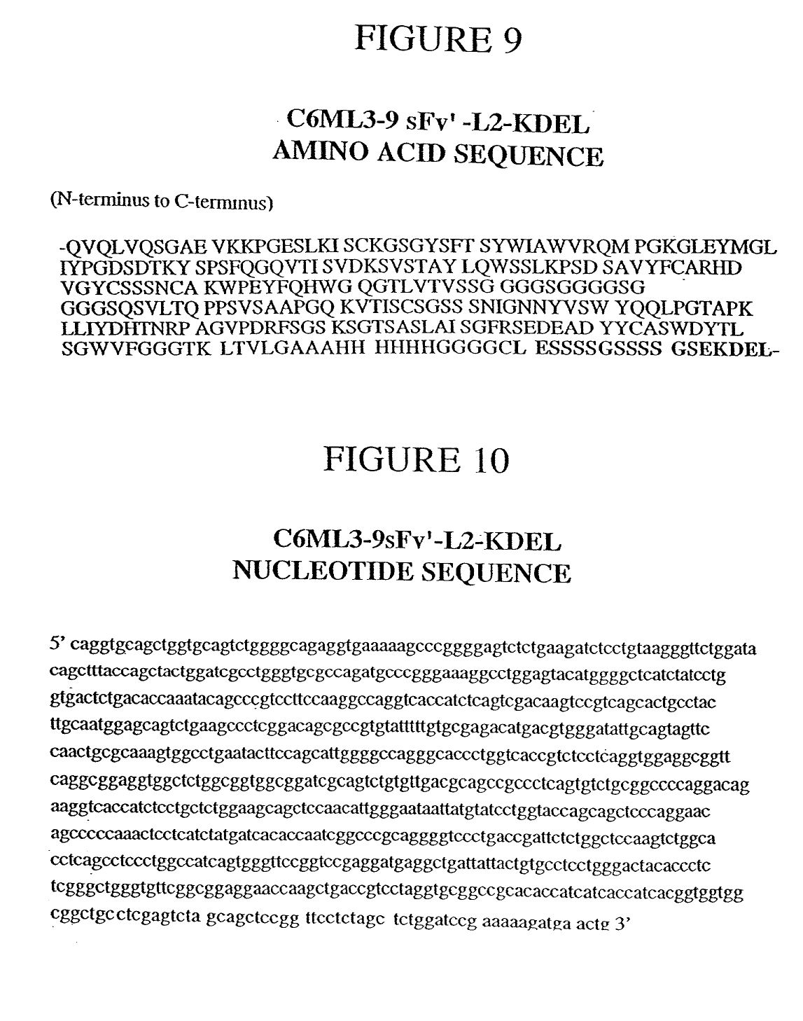

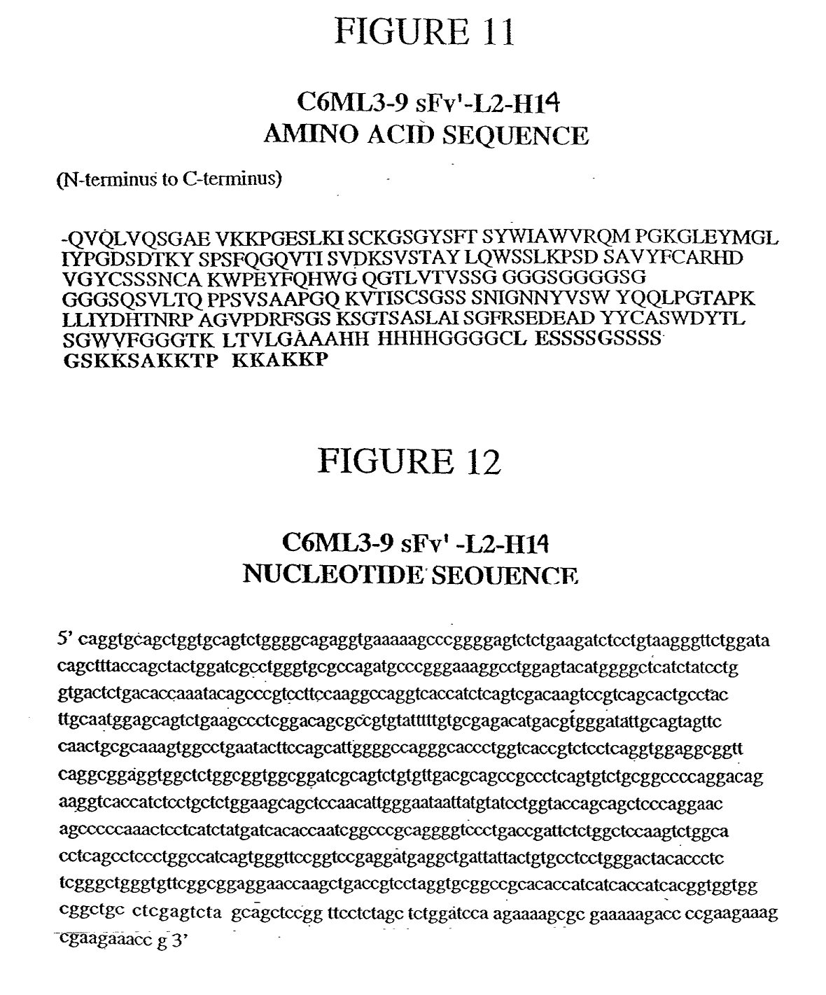

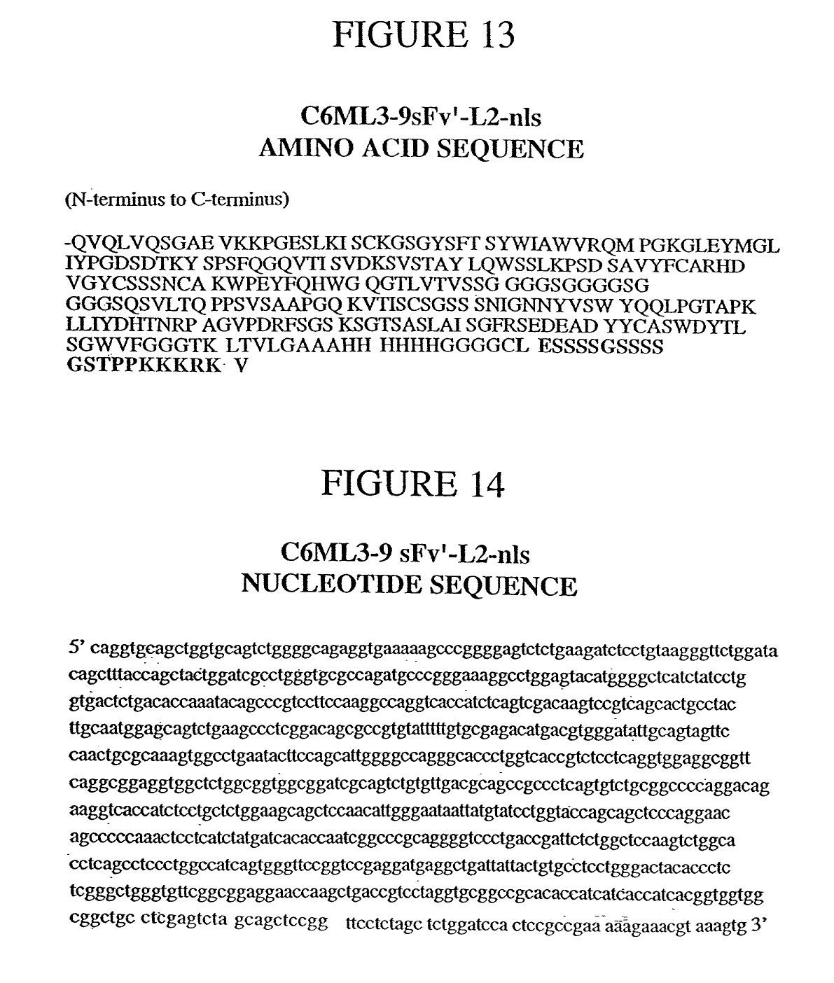

C6ML3-9 sFv′ single-chain binding protein constructs are listed below. The DNA/amino acid sequence of the fusion proteins could be found in FIGS. 7 to 14, respectively.

C6ML3-9 sFv′-L1-KDEL

C6ML3-9 sFv′-L2-KDEL

C6ML3-9 sFv′-L2-H14

C6ML3-9 sFv′-L2-nls

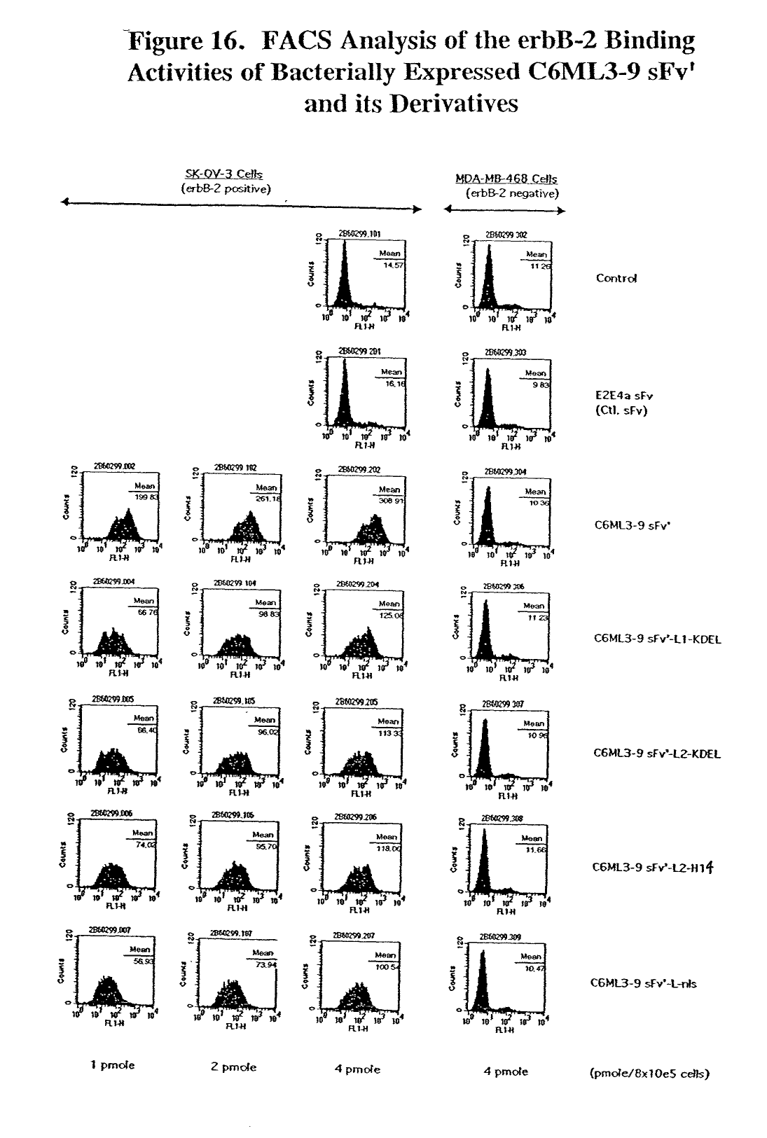

The above C6ML3-9 sFv′ derivatives as well as the parental C6ML3-9 sFv′ were all expressed in bacteria and purified (data not shown). The purified proteins were active in their erbB-2 binding activity as analyzed by FACS (see Example 9, FIG. 16).

### Example 3

Bacterial Production and Purification of C6ML3-9 sFv′

The example which follows describes the bacterial production and purification of C6ML3-9 sFv′.

A. Fermentation and inductions

A stab of frozen TG1 cells containing C6ML3-9 sFv′ plasmid (obtained by quickly scratching the frozen glycerol stock with a sterile pipet tip) was grown in 250 mL 2TY medium containing 2% glucose and 50 μg/mL carbenicillin in a 1 L flask at room temperature and 200 rpm for 16 hours.

The overnight culture was diluted 100-fold into 2 L flasks containing 750 mL 2TY medium+0.1% glucose+100 μg/mL ampicillin and grown to A₆₀₀˜1.5 at 37° C. and 200 rpm. Induction was performed with 0.5 mM IPTG at room temperature and 200 rpm for 16 hours.

The cells were harvested by centrifugation at 10,000 g for 10 minutes in 500 mL bottles. The supernatant was discarded after disinfection with Wescodyne and the cell pellet frozen at −70° C.

B. Purification of soluble C6ML3-9 sFv′

The frozen cells were placed on ice for 30 minutes. The cells were then resuspended by passage through a 60 cc syringe without a needle in osmotic shock buffer containing 200 mg/mL sucrose, 30 mM Tris-Cl, pH 8.0 and 1 mM EDTA using 25 mL buffer for each 1 L cell pellet. The cells were then stirred at 4° C. for 1 hour and centrifuged at 17000 g for 20 minutes.

The supernatant was saved and the cell pellet was resuspended in 5 mM MgSO₄ (made in distilled water) using 25 mL buffer for each 1 L cell pellet. The cells were then stirred at 4° C. for 1 hour and centrifuged at 17000 g for 20 minutes.

The supernatant was combined with the osmotic shock supernatant. If the mixture was viscous, it was sonicated with a tip sonicator for 5 minutes at 60% duty and setting 6. The sonicator used was Sonifier II Model 450 by Branson Ultrasonics. The mixture was then centrifuged at 17000 g for 30 minutes.

Dialysis tubing was prepared by cutting 12 inch pieces of 2000 molecular weight cut-off SpectraPor 7 dialysis membrane, rinsing extensively in distilled water and checking for leaks.

The cell lysate was loaded to 80% of the dialysis bag's capacity and dialyzed against a 10-fold excess of PBS at 4° C. Fresh PBS was added after one hour and dialysis continued at 4° C. overnight.

Fresh PBS was added and dialysis continued at 4° C. for one hour. If necessary, the pH and conductivity of the dialyzed lysate was checked to make sure they were within values for PBS. PBS has a pH value of 7.4 and conductivity≈18 ms.

Nickel-nitrilotracetic acid (Ni-NTA) agarose was prepared (Ni-NTA agarose from Qiagen, Catalog No. 30250) at 1 mL/L cell pellet by washing twice with 5 column volumes water and twice with 5 column volumes PBS in a batch format (in 50 mL conical tubes). Resin can be separated from wash buffer by centrifugation at 1200 rpm for 5 minutes in the Sorval T-21 centrifuge.

Imidazole was added to the dialyzed lysate to a final concentration of 20 mM and stirred with Ni-NTA resin at room temperature for 1 hour.

The lysate-resin mix was packed in a BIO-RAD low pressure column and the flow-through saved. The flow-through typically contained 10-15% uncaptured C6ML3-9 sFv′. The column was then washed with 10 column volumes PBS+35 mM Imidazole.

During the wash step, a 5 mL Q-Sepharose HiTrap column was attached to a 5 mL Heparin-Sepharose HiTrap column and the assembly was equilibrated with 50 mL PBS at 5 mL/min.

The bound protein was eluted in 2.5 column volumes PBS+250 mM Imidazole. 2 mL fractions were collected and the absorbance was read at 280 nm. The fractions with the highest absorbance were pooled.

The filtered protein was loaded immediately to the assembly of Q-Sepharose and Heparin-Sepharose columns at 5 mL/min. Do not store IMAC-purified protein at 4° C. overnight at contaminants may coprecipitate sFv′.

The flow-through was saved and the assembly was washed with 10 mL PBS. The wash was added to the flow-through. The HiTrap columns can be regenerated using 5 column volumes PBS+1 M NaCl followed by equilibration with 5 column volumes PBS. For long term storage, ethanol should be added to the PBS to 20%.

The purified C6ML3-9 sFv′ was dialyzed against 100-fold excess PBS at 4° C. overnight.

The C6ML3-9 sFv′ purification was analyzed by SDS-PAGE. Using spectrophotometric scans to ascertain the concentration of C6ML3-9 sFv′. For A₂₈₀=1 assume a concentration of 0.7 mg/mL C6ML3-9 sFv′.

The C6ML3-9 sFv′ was stored at 4° C. with 0.02% sodium azide. For long term storage, C6ML3-9 sFv′ was quick frozen in a dry-ice/ethanol bath followed by storage at −70° C.

### Example 4

Preparing C6ML3-9 sFv′ for Chemical Conjugation with Protamines

C6ML3-9 sFv′ and its derivative proteins may be prepared for chemical conjugation essentially as described in the following example.

A. Concentration of C6ML3-9 sFv′

Millipore Centriplus-10 centrifugal concentrators (10 kD MWCO, 15 mL capacity, 3000 g max) were used to concentrate C6ML3-9 sFv′. Concentration is significantly faster at 8° C.-10° C. than at 4° C.

Following centrifugation, C6ML3-9 sFv′ was generally available at a concentration of 1.5-2 mg/mL. Once C6ML3-9 sFv′ concentration approached 7-8 mg/mL the operation of the concentration devices slowed significantly and it took up to several hours to concentrate C6ML3-9 sFv′ over 10 mg/mL. When possible, C6ML3-9 sFv′ was concentrated to 10-15 mg/mL.

During concentration, the required number of disposable PD-10 Sephadex G-25 columns were equilibrated with 25 mL 0.1 M Na phosphate pH 8.0+1 mM EDTA.

If concentration polarization occurred, that is, a film of protein formed just above the membrane at 10-15 mg/mL, the film was thoroughly disrupted (without foaming) for 80-90% C6ML3-9 sFv′ recovery. A final rinse with small amounts of PBS was useful in further improving C6ML3-9 sFv′ recovery.

A spectrophotometric scan allowed quantitation of C6ML3-9 sFv′ concentration.

B. Reduction of the terminal sulfhydryl of C6ML3-9 sFv′

To C6ML3-9 sFv′ present at 10-15 mg/mL, DTT was added to a final concentration of 1 mM. The C6ML3-9 sFv′ were then mixed and incubated at room temperature for 30 minutes.

2.5 mL reduced protein was loaded per PD-10 desalting column. The flow-through was discarded and 3.5 mL 0.1 M Na phosphate pH 8.0 was added. The eluent was collected in a clean 50 mL conical tube.

The reduced C6ML3-9 sFv′ was diluted 5 or 10-fold in 500 μL 0.1 M Na phosphate pH 8.0. Using 0.1 M Na phosphate pH 8.0 as the blanking buffer A₂₈₀ of the reduced protein was measured and the sFv′ concentration estimated (when A₂₈₀=1.0, sFv′ concentration is 0.7 mg/mL, assuming C6ML3-9 sFv′ has a molecular weight of about 28193 Da. The cuvette containing diluted C6ML3-9 sFv′ was zeroed at 412 nm. One μL of a 50 mM stock solution of DTNB made in pure ethanol was added, mixed well, and measured at A₄₁₂. The reading took 2-3 minutes to stabilize. The background A₄₁₂ was also measured by adding 1 μL DTNB to 500 μL 0.1 M Na phosphate pH 8.0+1 mM EDTA. The number of reduced sulfhydryls per C6ML3-9 sFv′ was quantitated using the extinction coefficient of 13600 M−1 cm−1 for the free thionitrobenzoic acid anion (if a one molar solution of C6ML3-9 sFv′ has exactly one reduced sulfhydryl per molecule then at pH 8 the A₄₁₂ is 13600). For C6ML3-9 sFv′, this number is 1.8.

By conducting the reoxidation at pH 8.2 in 0.2 M Tris buffer, it was found that the reoxidation of the intrachain disulfide occurs in about 4 hours, while the C-terminal sulfhydryl remained reduced. The procedure can also be done in less buffered conditions, for example, 0.01 M Tris, or phosphate buffered saline +0.01 M Tris buffer, which could weakly buffer at pH 8.2 as well as near neutrality.

### Example 5

Formation of C6ML3-9 sFv′-salmon Protamine Conjugate

A heterobifunctional linker, Sulfo-SMCC (Pierce Cat. No. 22322) was used to couple salmon protamine (Grade X, Sigma) via its alpha amino terminal group to the C-terminal sulfhydryl of C6ML3-9 sFv′.

A 10 mg/mL solution of salmon protamine sulfate was prepared in PBS. 50 mg Sulfo-SMCC was dissolved in this solution (Sulfo-SMCC is soluble up to 1 mM or ˜5 mg/mL in aqueous buffer). The reaction was then mixed and incubated at 37° C. for 30 minutes with intermittent mixing.

Linker-conjugated protamine was purified on a HiTrap Heparin-Sepharose column (alternative methods for purification include dialysis, desalting or size-exclusion chromatography).

A Bio-Rad protein assay (Catalog No. 500-0006, BioRad) was used to both determine protamine-rich fractions as well as to estimate their concentration. The most concentrated fractions were pooled but not dialyzed. The maleimide group on Sulfo-SMCC is stable at pH 7.4, 4° C. for 64 hours. If necessary linker-protamine conjugates were stored at −70° C.

C6ML3-9 sFv′ containing a single sulfhydryl per molecule was prepared by air oxidizing the DTT-reduced sFv′ in Example 4 at 4° C. until DTNB reaction showed presence of one free sulfflydryls per sFv′ molecule (typically 24-73 hours). At pH 8.2, it reoxidizes to the single-SH state in about 4 hours.

The amount of 1 M sodium phosphate monobasic needed to adjust the pH of 10 mL 0.1 M sodium phosphate solution from 8 to 7 was determined experimentally. The amount needed for the volume equal to that of sFv′ solution was calculated and the required amount of 1 M sodium phosphate monobasic was added to bring the C6ML3-9 sFv′ solution to pH 7.

To react the linker-protamine conjugate with reduced C6ML3-9 sFv′, linker-protamine conjugate from above at a ratio of 5 moles protamine/mole C6ML3-9 sFv′ was added to a solution of single-sulfhydryl C6ML3-9 sFv′ at 2-5 mg/mL. This solution was then mixed and incubated at room temperature for 2 hours.

Size-exclusion chromatography on a Superose 12 column was used to remove unreacted protamine. Fractions were collected in 2 mL polypropylene tubes and analyzed by SDS-PAGE.

Fractions containing C6ML3-9 sFv′-protamine conjugates were pooled and passed through a HiTrap Heparin-Sepharose column.

The column was washed and bound protein eluted with PBS+2M NaCl. The fractions were analyzed and those fractions containing fusion protein were pooled.

The pooled fractions were dialyzed against PBS and store at 4° C. with 0.02% azide or at −70° C. for long-term storage.

### Example 6

Formation of C6ML3-9 sFv′ Human Histone H1 and C6ML3-9 sFv′ Human Protamine P1conjugates

An H1 peptide, comprising residues 166 to 192 of human histone H1 (AKKAKSPKKAKAAKPKKAPKSPAKAK) was synthesized by solid phase synthesis and coupled to maleimide on its terminal amino group. C6ML3-9 sFv′, at a concentration of 5-15 mg/ml⁻¹, and bearing one free SH per protein, was reacted with a ten-fold molar excess of maleimide-H1. this reaction was performed under gentle stirring for 2 hours at room temperature, protected from light, and in 100 mM phosphate buffer pH 7.4. Excess H1 peptide was removed from the reaction mix by ultrafiltration on 10 kDa polyethersulfone membrane (Pall Filtron).

The C6ML3-9-P1 conjugate was synthesized and purified similarly using maleimide-P1 as starting material. The P1 synthetic peptide, consisting in the residues 11 to 28 of the human protamine (SRSRYYRQRQRSRRRRRR) was synthesized by solid phase synthesis and coupled to maleimide on its terminal amino group.

### Example 7

Synthesis and Purification of C6ML3-9-PEG-(C₁₈)₂

The example which follows describes preparation of a single-chain binding polypeptide (C6ML3-9 sFv′) coupled to a lipid-associating moiety, PEG-(C₁₈)₂.

In order to formulate targeted liposomes C6ML3-9 sFv′ was coupled to a lipid bearing 2 palmitic acid chains, with a polyethylene glycol (PEG) spacer. This synthesis was done by coupling maleimide-PEG-(C₁₈)₂ to the side chain sulfhydryl group of C6ML3-9 sFv′.

To prepare maleimide-PEG-(C₁₈)₂ diglycolic anhydride was reacted with dioctadecylamine to produce dioctadecyl-carbamoyl-methoxy-acetic acid. This product was reacted with Boc-NH-PEG-NH₂ and unprotected to form ((2-amino-PEG-ethylcarbamoyl)-methoxy)-N,N-dioctadecyl-acetamide [NH2-PEG-(C₁₈)₂]. Maleimido-propionic acid was then added to the terminal NH₂ of PEG to yield maleimide-PEG-(C₁₈)₂. Maleimide-PEG-(C₁₈)₂ was finally reacted with C6ML3-9 sFv′ (10 moles of maleimide-PEG-(C₁₈)₂/1 mole of C6ML3-9 sFv′ bearing 1.07 SH per protein) to form C6ML3-9-PEG-(C₁₈)₂.

The C6ML3-9-PEG-(C₁₈)₂ conjugate was purified by reverse phase HPLC (0.1% TFA, 0-100% acetonitrile, Vydac 214TP54 C₄ column). The product analyzed by SDS-PAGE and silver staining was showed to be pure, without detectable contaminating compound. The C6ML3-9-PEG-(C₁₈)₂ conjugate was lyophilized in order to remove solvents and TFA, solubilized in H₂O, and stored at −80° C.

### Example 8

FACS Analysis of erbB-2 Binding Activity of the Anti-erbB-2 C6ML3-9 sFv′ and Their Salmon Protamine Conjugates

In order to conduct a cell surface anti-erbB-2 sFv′ binding assay, SK-OV-3, a human ovarian cancer cell line expressing erbB-2 (ATCC, Catalog No. HTB-77) was used as the positive cell line and MDA-MB-468 (ATCC, Catalog No. HTB-132) as the negative cell line. 8×10⁵ cells were used for each FACS sample. Cells were first incubated in 200 μl primary antibody solution, which contains indicated amounts of either anti-erbB-2 sFv′, its conjugate to salmon protamine, or the sFv′ fusion derivatives at 4° C. for 1.5-2 hours. Upon rinsing with PBS, rabbit anti-His polyclonal antibody was used as secondary antibody (Santa Cruz Cat. #sc-803, 200 ug/ml), followed by goat anti-rabbit IgG FITC conjugate as tertiary antibody (Sigma F-0511). Cells were fixed in 200 μl of 2% paraformaldehyde (PFA)/PBS at 4° C. for 30 minutes prior to FACS analysis on FACScan. The sample named “control” used PBS instead of the sFv′ and the sample named E2E4a was an irrelevant sFv control.

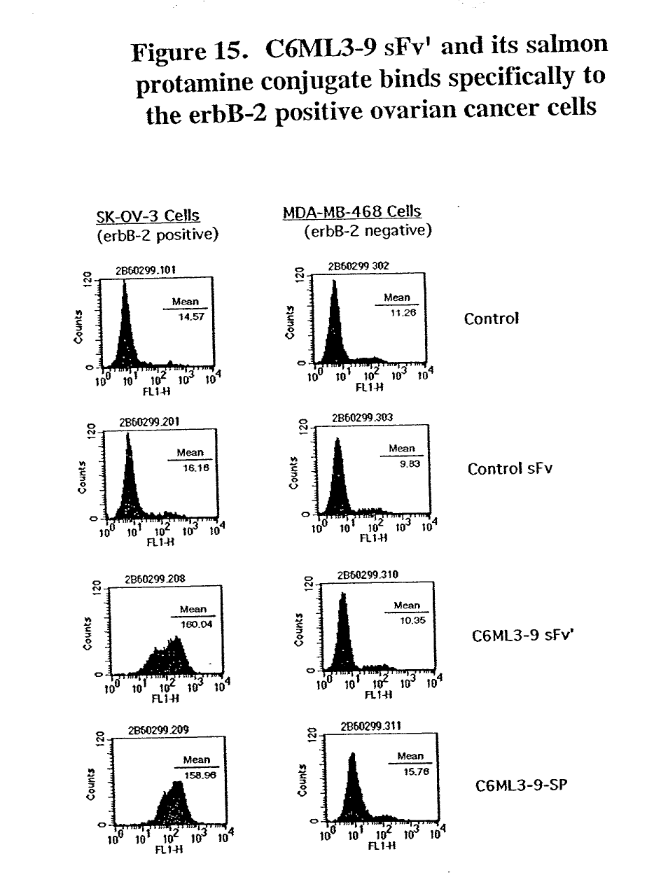

FIG. 15 shows that C6ML3-9 sFv′ (4 pmole) specifically binds to the erbB-2 positive SK-OV-3 cell line but not the erbB-2 negative MDA-MB468 cell line. The salmon protamine conjugate, C6ML3-9-SP, retains its erbB-2 binding specificity.

FIG. 16 is the result of a FACS analysis on the purified C6ML3-9 sFv′ fusion derivatives, which shows that all the C6ML3-9 sFv′ fusion derivative proteins also binds erbB-2 specifically in a dose responsive manner.

### Example 9

Interaction of Plasmid DNA with the anti-erbB-2 sFv′-salmon Protamine Conjugates

The ability of the anti-erbB-2 C6ML3-9 sFv′-salmone protamine (SP) conjugates to complex with plasmid DNA was tested by a gel mobility shift analysis.

A. Materials

200 ng plasmid DNA (pGL-control (Promega) or pXL3031)

1.45 pmole (=45.5 ng) C6ML3-9 sFv′-SP, C6.5 sFv′-SP, unconjugated C6ML3-9 sFv′ or C6.5 sFv′ control in PBS, 2 fold increase up to 11.6 pmole

1×PBX (Gibco) make up the reaction volume to 20 ul

B. Procedure

The DNA was added last, and the mixture incubated on ice for 1 to 1.5 hour (in the case of kinetics studies, incubation time was from 5 minutes to 60 minutes as indicated). 2 μl of loading buffer (50% glycerol in 1×TE with dye) was added to the 20 μl reaction. The reaction was electrophoresed on 0.8% agarose gel in 1×TAE, 150 V for about an hour at room temperature and stained with EtBr overnight.

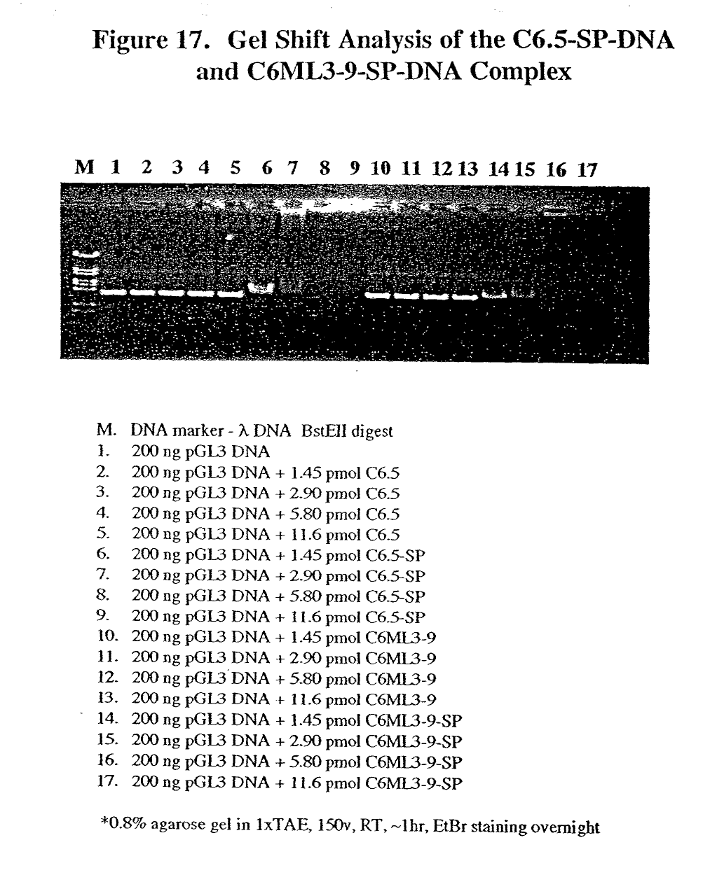

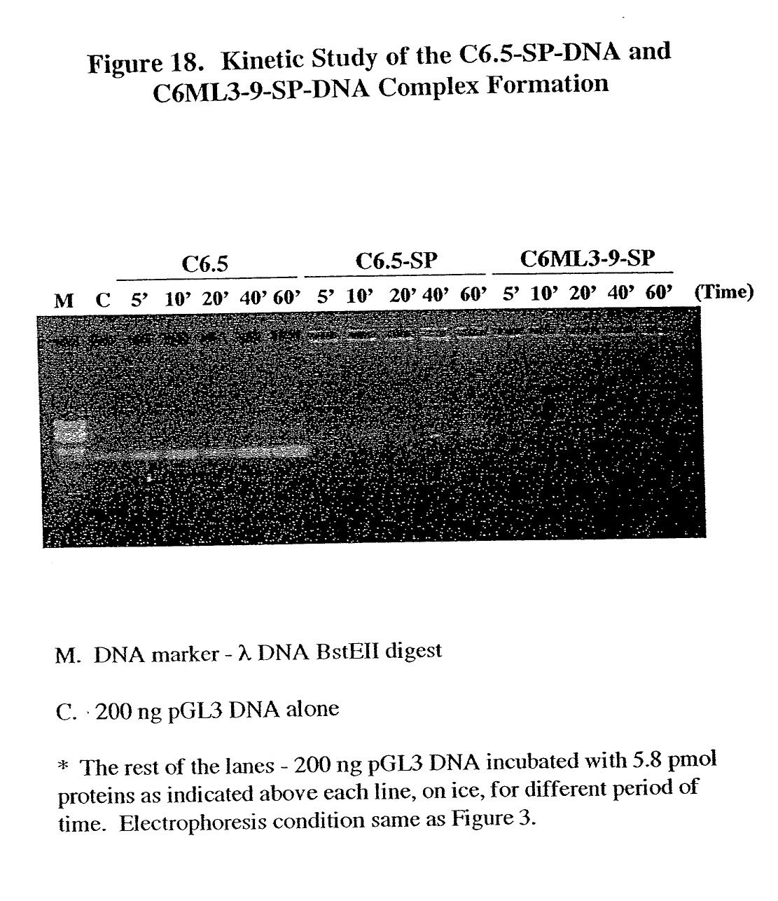

With 2.9 pmole (about 90 ng) C6.5 sFv′-SP or C6ML3-9 sFv′-SP, retardation of the plasmid DNA (200 ng) band was observed (FIG. 17). With 5.8 pmole (360 ng) C6.5 sFv′-SP or C6ML3-9 sFv′-SP, the complex could form in 5 minutes (FIG. 18). However, the complexes formed in 30 minutes did not give optimal transfection data, indicating more time might be needed for compaction of the complex.

### Example 10

Reporter Plasmid Gene Delivery to erbB-2 Positive Cells by the Anti-erbB-2 sFv′-salmon Protamine Conjugates

A. Delivery of luciferase gene

Gene delivery experiments were carried out with the anti-erbB-2 sFv′-[salmon protamine]-DNA complex (C6.5 sFv′-SP-DNA or C6ML3-9 sFv′-SP-DNA). The reporter DNA plasmid was the pGL3-control from Promega, which encodes the luciferase gene under control of the SV40 early promoter and enhancers. The erbB-2 positive cell line used in the study was SK-OV-3, a human ovarian cancer cell line. 200 ng of pGL3 reporter plasmid DNA was incubated with increasing amounts of either the sFv′-[salmon protamine] conjugates (sFv′-SP), or the sFv′ mixed with salmon protamine (SP) alone as described. Formation of the protein-DNA complex was confirmed by gel mobility shift analysis (data not shown). The mixture of protein and DNA were then incubated with SK-OV-3 cells in the absence or presence of 100 μM chloroquine. The protein-DNA mixture was removed from the cell culture after a 20 hour incubation. Cells were harvested for luciferase assays at about 40 hours post-incubation using a Dynex MLX Luminometer. The experiment data presented are an averaged data from quadruplet samples of a typical experiment.

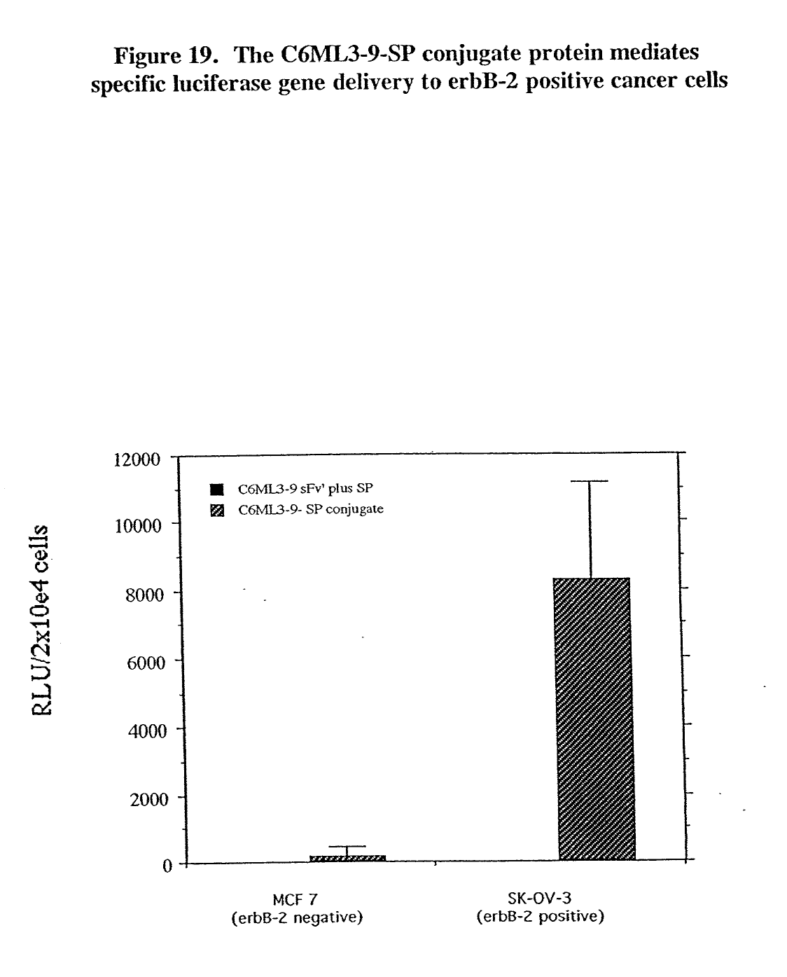

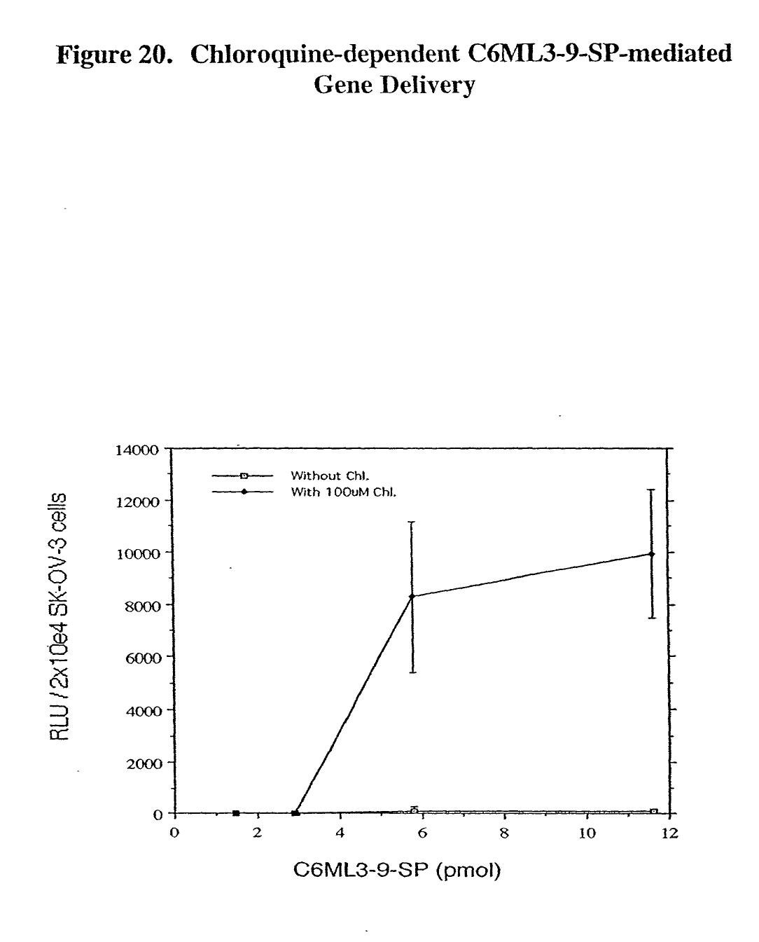

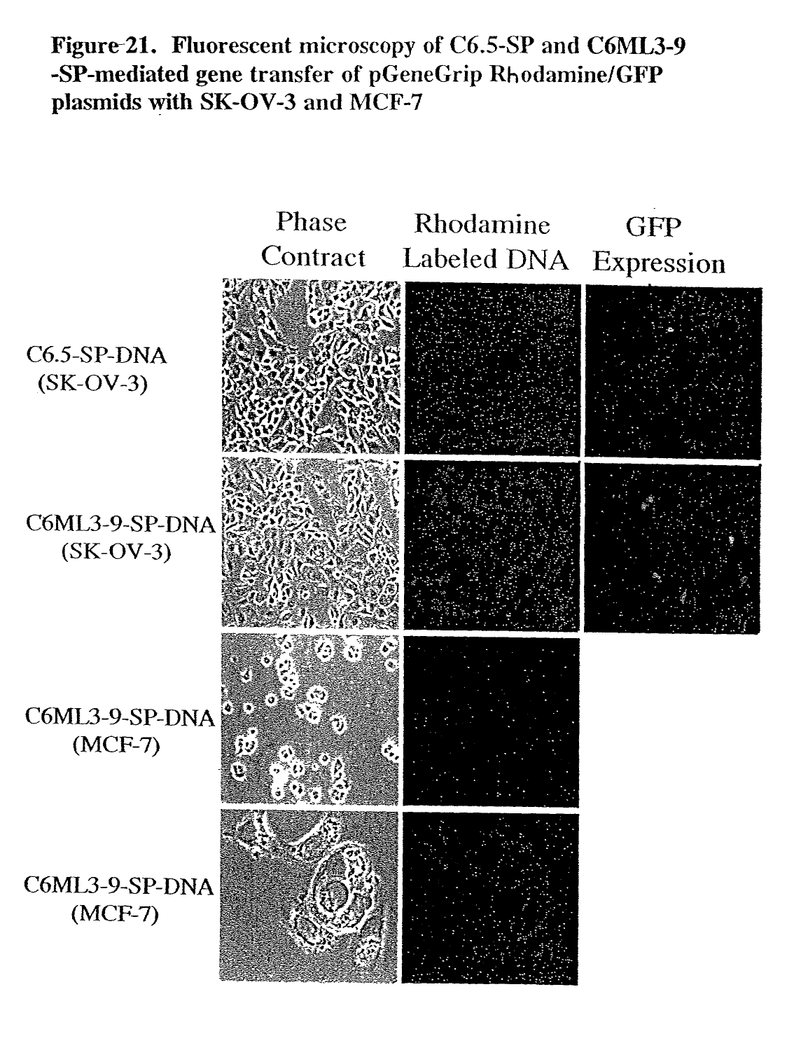

FIG. 19 is an example of the non-viral gene delivery experiments using C6ML3-9 sFv′-SP-DNA complexes, showing that (1) the C6ML3-9 sFv′-[salmon protamine] conjugate delivered luciferase reporter plasmids into SK-OV-3 cells, while the sFv′ mixed with salmon protamine (no covalent bond between the sFv′ and SP) did not; and (2) the C6ML3-9 sFv′-SP-mediated luciferase gene delivery was erbB-2 dependent as evidenced by minimal luciferase activity observed in MCF-7 cells (erbB-2 negative control, ATCC, Catalog No. HTB-22). The delivery specificity could be further confirmed by the fact that the C6ML3-9 sFv′-SP-mediated luciferase gene delivery to SK-OV-3 cells could be competed away by pre-incubating the cells with free C6ML3-9 sFv′ (data not shown). FIG. 20 demonstrates that the C6ML3-9 sFv′-SP mediated luciferase gene delivery to SK-OV-3 cells are chloroquine-dependent. C6.5 sFv′-SP was able to mediate specific luciferase gene delivery to erbB-2 positive SK-OV-3 cells, although with lower efficiency as compared to C6ML3-9 sFv′-SP (data not shown and FIG. 21).

B. Delivery of Rhodamine-labeled pGeneGrip Reporter Plasmid Encoding Green Fluorescent Protein (GFP)

pGeneGrip Rhodamine/GFP plasmid (Gene Therapy Systems) was used as another reporter plasmid for studying C6.5 sFv′-SP and C6ML3-9 sFv′-SP-mediated gene delivery. In this case, plasmid DNA encoding green fluorescent protein (GFP) was labeled with rhodamine, which allows one to follow internalization of the plasmid DNA as well as the expression of GFP. This reporter facilitated evaluation of the gene delivery efficiency at both DNA and protein expression levels. The formation of protein/DNA complexes between either C6.5 sFv′-SP or C6ML3-9 sFv′-SP and pGeneGrip plasmid DNA were confirmed by gel mobility shift analysis (data not shown). SK-OV-3 and MCF-7 cells were incubated with the protein/DNA complexes and fixed at 6, 24, 48, and 72 hours post-incubation for fluorescent microscopy. FIG. 21 represents the data from the 48 hour time point. While no rhodamine fluorescence was observed with sFv′ or salmon protamine alone (data not shown), it is clear that C6ML3-9 sFv′-SP-mediated gene delivery had an efficiency of over 80% at the DNA level, which was higher than the C6.5 sFv′-SP. The rhodamine labeled DNA could be seen inside of SK-OV-3 cells at 24 hours (data not shown). However, the GFP gene expression, was very low, about 1-2% cells being GFP positive in the case of C6ML3-9 sFv′-SP-mediated delivery at 48 hours. It should be noted that, although low, GFP expression level still correlates with the amount of DNA inside of the cells (FIG. 21, compare C6.5 sFv′-SP-DNA with that C6ML3-9 sFv′-SP-DNA). Furthermore, no additional GFP expression was observed with 72 hour time point (data not shown). The low expression of GFP may be caused by the difficulty of plasmid DNA either escaping from the endosomes or reaching the nucleus. No GFP expression was observed with the control MCF-7 cells. Under higher magnification, the low amounts of rhodamine-labeled DNA associated with MCF-7 cells were found to be mainly on the surface.

### Example 11

Transfection of 3T3 and 3T3-HER2 Cell Lines

Transfections were done using C6.5-H1, C6ML3-9 sFv′-H1, C6ML3-9 sFv′-P1 (comprising C6ML3-9 coupled to human protamine P1 peptide) and C6ML3-9 sFv′-salmon protamine (C6ML3-9-SP). Conjugates were mixed in 20 nM NaCl with pXL3031 (pCOR Luc⁺) reporter plasmid at different ratios and, after a 10 minute incubation, used to transfect c-erbB-2 expressing (3T3-HER2) or non-expressing (3T3) cell lines. Transfection were performed in the presence of 10% fetal calf serum (FCS) for 3T3-HER2, or 10% calf serum (CS) for 3T3. After 24 hours of incubation, cells were washed twice with PBS and lysed with 200 μl of cell culture lysis reagent (Promega). Luciferase expression was quantified using a luciferase assay kit (Promega) and a Lumat LB9501 luminometer (EG and G). Light emission (RLU) was normalized to the protein concentration of each sample, measured using the Pierce BCA assay. Conditions of transfection are summarized below for each experiment.

The results show that all tested conjugates are able to transfect c-erbB-2 positive cells.

TABLE 2

| | | RLU/μg |
|---|---|---|
| 3T3 | Transfection Conditions | of cell proteins |
| RPR120535 | 6 nmoles/μg of DNA, no | 26 100 000 |
| (control) | chloroquine | (±2 160 000) |
| C6.5-H1 | 7 μg/μg of DNA, no chloroquine | 6 (±7) |
| C6ML3-9 sFv′- | 7 μg/μg of DNA, no chloroquine | 0 (±0) |
| H1 | | |
| C6ML3-9 sFv′- | 6 μg/μg of DNA, 150 μM | 9 (±15) |
| P1 | chloroquine | |
| C6ML3-9 sFv′- | 4 μg/μg of DNA, 200 μM | 1080 (±715) |
| SP | chloroquine | |

Table 2 shows the comparison of transfection efficiencies of C6.5-H1, C6ML3-9 sFv′-H1, C6ML3-9 sFv′-P1, C6ML3-9 sFv′-SP in 3T3 cells. All transfections were done in the presence of 10% serum. Best transfection conditions are indicated for each compound. All complexes with sFv′ conjugates were formed in 20 mM NaCl, and all complexes with RPR120535 were formed in 20 mM NaHCO₃ 150 mM NaCl. Values correspond to the mean of three different measures of the same assay.

TABLE 3

| 3T3-HER2 | Transfection Conditions | RLU/μg of cell proteins |
|---|---|---|
| RPR120535 | 6 nmoles/μg of DNA, no | 2 980 000 |
| (control) | chloroquine | (±271 000) |
| C6.5-H1 | 7 μg/μg of DNA, no chloroquine | 659 (±240) |
| C6ML3-9 | 7 μg/μg of DNA, no chloroquine | 27 400 (±6030) |
| sFv′-H1 | | |
| C6ML3-9 | 6 μg/μg of DNA, 150 μM | 10 024 (±3757) |
| sFv′-P1 | chloroquine | |
| C6ML3-9 | 4 μg/μg of DNA, 200 μM | 220 000 (±20 000) |
| sFv′-SP | chloroquine | |

Table 3 shows the comparison of transfection efficiencies of C6.5-H1, C6ML3-9 sFv′-H1, C6ML3-9 sFv′-P1, C6ML3-9 sFv′-SP in 3T3-HER2 cells. All transfections were done in the presence of 10% serum. Best transfection conditions are indicated for each compound. All complexes with sFv′ conjugates were formed in 20 mM NaCl, and all complexes with RPR120535 were formed in 20 mM NaHCO₃ 150 mM NaCl. Values correspond to the mean of three different measures of the same assay.

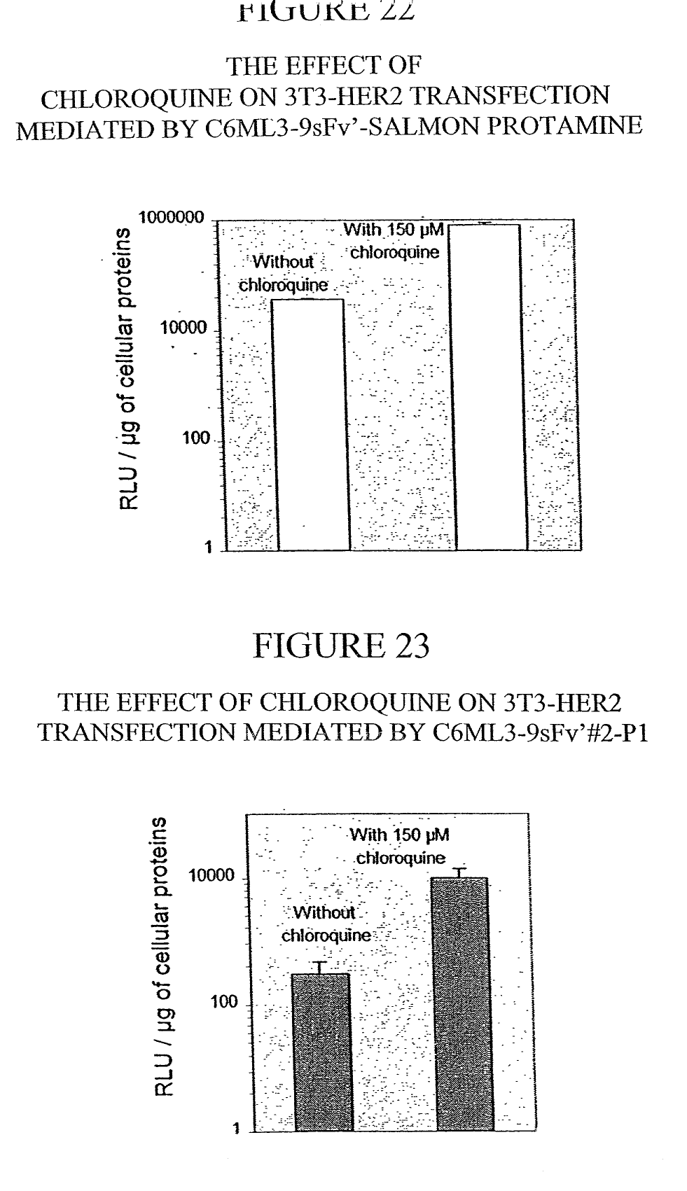

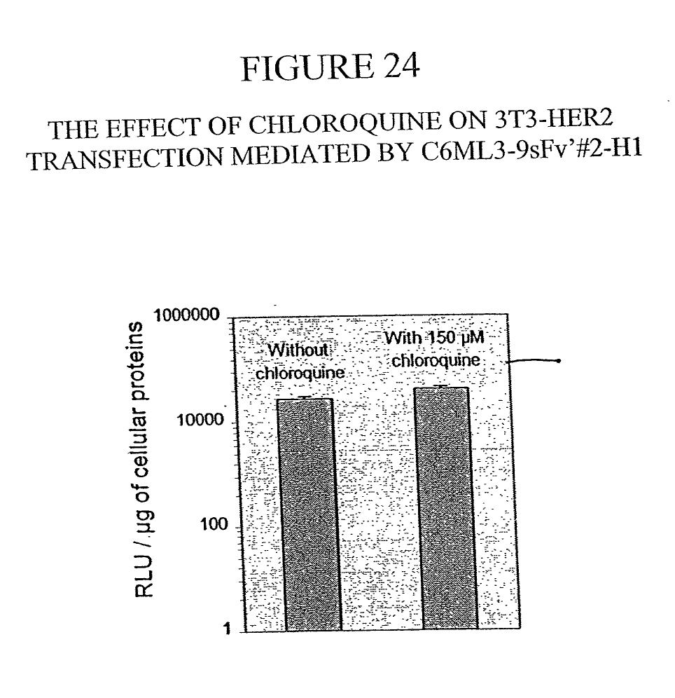

FIGS. 22, 23, and 24 are bar graphs illustrating the effect of chloroquine on 3T3-HER2 transfection mediated by sFv′-peptide conjugates.

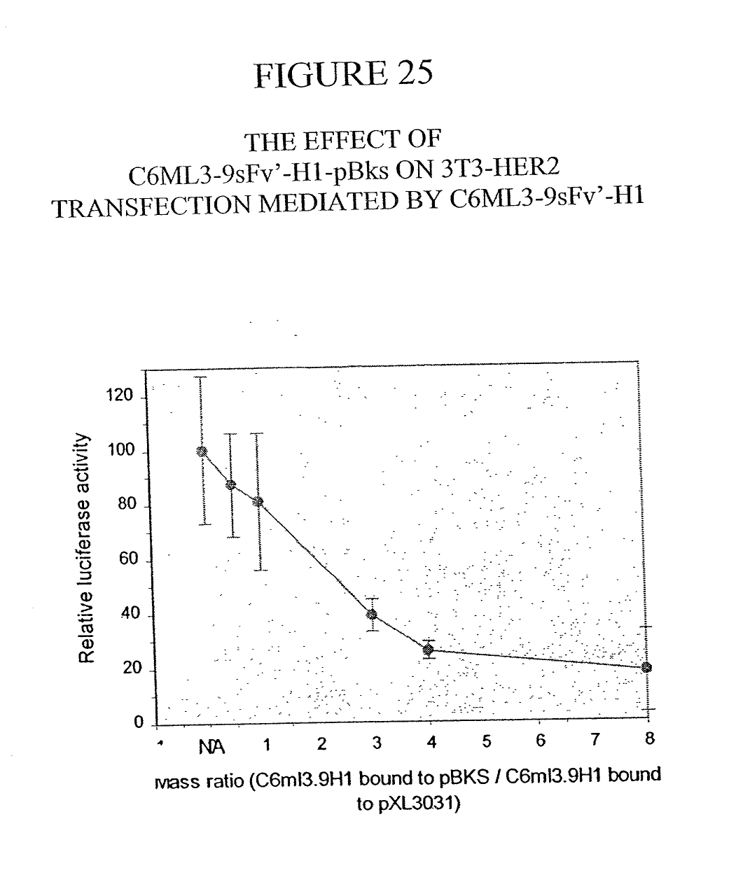

FIG. 25 is a graph which illustrates the effect of C6ML3-9 sFv′-H1-pBks on 3T3-HER2 transfection mediated by C6ML3-9 sFv′-H1. The DNA to protein mass ratio was 1:7 for both complexes.

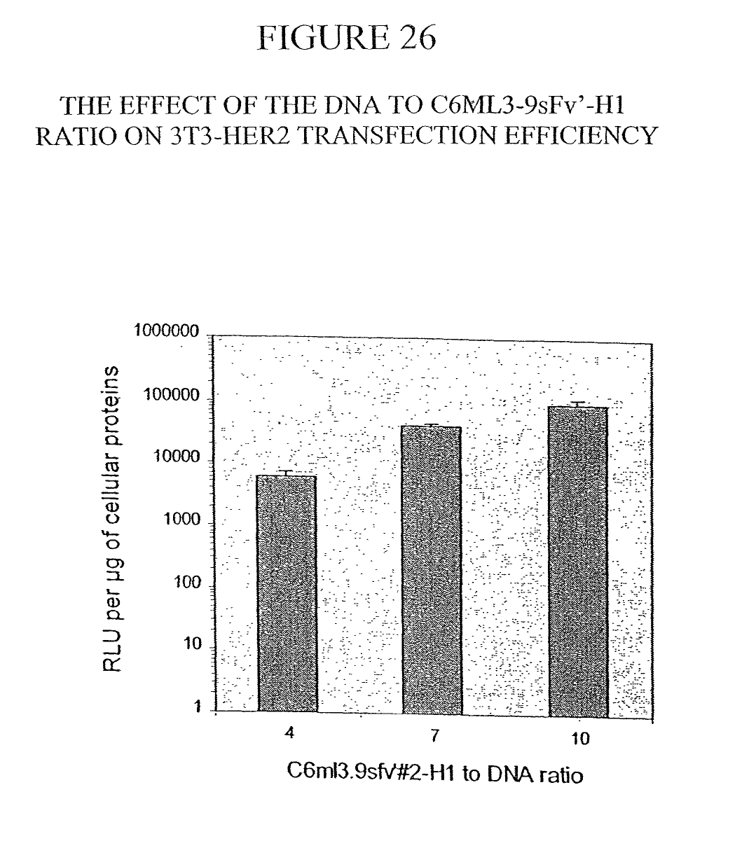

FIG. 26 is a graph which illustrates the effect of the DNA to C6ML3-9 sFv′-H1 ratio on 3T3-HER2 transfection efficiency. The graph illustrates that increasing the C6ML3-9 sFv′-H1 to DNA mass ratio from 4 to 10 resulted in a 10-fold increase in transfection efficiency.

The transfection activity of C6ML3-9 sFv′-H1 could be reduced by addition to the transfection medium of either free C6ML3-9 sFv′ or C6ML3-9 sFv′-H1 complexed to pBks plasmid demonstrating the specificity of gene transfer.

1. A gene-delivery compound comprising: (A) a single-chain binding polypeptide having at least one effector segment which includes at least one cysteinyl residue; and (B) a nucleic acid-binding moiety which is coupled to said polypeptide by said residue.

2. The compound of claim 1 having a binding region which is effective in binding a surface marker of a mammalian cell wherein said binding region comprises a single-chain Fv protein.

3. A composition comprising the compound of claim 1 and a nucleic acid associated reversibly with said moiety.

4. The compound of claim 1 wherein said polypeptide is effective in binding a surface marker of a mammalian cell.

5. The compound of claim 4 wherein said marker is a tumor antigen.

6. The compound of claim 5 wherein said marker is selected from the group consisting of erbB-2, erbB-3, erbB-4, p53, p21 ras, transferrin receptor, Lewis Y antigen, carcinoembryonic antigen, epidermal growth factor, MUC1, and any other tumor-associated or tumor-specific antigen.

7. The compound of claim 1 wherein said nucleic acid-binding moiety is selected from the group consisting of salmon protamine, subfragments of salmon protamine, human histone H1, subfragments of human histone H1, human protamine, subfragments of human protamine, HMG, polylysine or any other DNA binding polypeptide.

8. The compound of claim 7 wherein the nucleic acid-binding moiety is salmon protamine.

9. The compound of claim 1 further comprising an additional effector segment that binds reversibly with nucleic acids.

10. The compound of claim 1 further comprising an additional effector segment that facilitates endosomal escape or avoidance.

11. The compound of claim 1 further comprising an additional effector segment that facilitates non-endosomal transport in a cell.

12. The compound of claim 1 further comprising an additional effector segment that facilitates entry into the nucleus of a targeted cell.

13. The compound of claim 9 wherein said additional effector segment is a human histone H1 peptide sequence.

14. The compound of claim 10 wherein said additional effector segment comprises the carboxyl-terminal sequence that binds to the KDEL receptor in the Golgi, SEKDEL.

15. The compound of claim 12 wherein said additional effector segment comprises the SV40 large T antigen nuclear localization sequence, TPPKKKRKV.

16. The compound of claim 1 further comprising at least one spacer sequence.

17. The compound of claim 16 further comprising at least one spacer sequence located between said effector segment containing said cysteinyl residue and an additional effector segment.

18. The compound of claim 17 wherein said spacer sequence comprises at least one (Ser₄Gly) or (Gly₄Ser) segment.

19. The compound of claim 18 comprising two (Ser₄Gly) or (Gly₄Ser) segments.

20. The compound of claim 1 including a heterobifunctional crosslinking agent which couples said cysteinyl residue to said nucleic acid-binding moiety.

21. The compound of claim 20 wherein said heterobifunctional crosslinking agent is selected from the group consisting of succinimidyl trans-4(maleimidylmethyl)-cyclohexane-1-carboxylate (SMCC) and sulfoSMCC.

22. The composition of claim 3 wherein said nucleic acid comprises DNA encoding a therapeutic gene.

23. The composition of claim 22 wherein said therapeutic gene is a lymphokine.

24. The composition of claim 22 wherein said therapeutic gene is tumor necrosis factor.

25. The composition of claim 22 wherein said therapeutic gene is an intrabody.

26. The composition of claim 22 wherein said therapeutic gene is selected from the group consisting of tumor suppressor genes, p53, proapoptotic genes, suicide genes, prodrug converting genes, HSV-TK and anti-angiogenic genes.

27. The compound of claim 1 comprising C6ML3-9 sFv′-H1.

28. The compound of claim 1 comprising C6ML3-9 sFv′-P1.

29. The compound of claim 1 comprising C6ML3-9 sFv′-SP.

30. A gene-delivery compound comprising: (A) a single-chain binding polypeptide having at least one effector segment which includes at least one cysteinyl residue; and (B) a lipid-associating moiety which is coupled to said polypeptide by said residue.

31. A gene-delivery composition comprising the compound of claim 30 and a liposome in association with said lipid-associating moiety.

32. The composition of claim 31 further comprising a nucleic acid in association with said liposome.

33. The compound of claim 30 wherein said polypeptide is effective in binding a surface marker of a mammalian cell.

34. The compound of claim 33 wherein said marker is a tumor antigen.

35. The compound of claim 34 wherein said marker is selected from the group consisting of erbB-2, erbB-3, erbB-4, p53, p21 ras, transferrin receptor, Lewis Y antigen, carcinoembryonic antigen, epidermal growth factor, MUC1, and any other tumor-associated or tumor-specific antigen.

36. The compound of claim 30 wherein said lipid-associating moiety is selected from the group consisting of linear, branched, cyclic, and polycyclic compounds capable of insertion into and retention of lipid-containing compositions.

37. The compound of claim 36 wherein said moiety contains polyethylene glycol (PEG).

38. The compound of claim 36 wherein said moiety is maleimide-PEG-(C₁₈)₂.

39. The compound of claim 37 wherein the PEG portion of said maleimide-PEG-(C₁₈)₂ has about 10 to about 100 oxyethyl units.

40. The composition of claim 31 wherein said polypeptide is located on the surface of said liposome.

41. The composition of claim 31 wherein said liposome is a stealth liposome.

42. The compound of claim 30 further comprising an additional effector segment capable of associating with nucleic acid.

43. The compound of claim 30 further comprising an additional effector segment that facilitates endosomal escape.

44. The compound of claim 30 further comprising an additional effector segment that facilitates non-endosomal transport in the cell.

45. The compound of claim 30 further comprising an additional effector segment that facilitates entry into the nucleus of a targeted cell.

46. The compound of claim 42 where in said additional effector segment comprises a human histone H1 peptide sequence.

47. The compound of claim 43 wherein said additional effector segment comprises the carboxyl-terminal sequence that binds to the KDEL receptor in the Golgi, SEKDEL.

48. The compound of claim 45 wherein said additional effector segment comprises the SV40 large T antigen nuclear localization sequence, TPPKKKRKV.

49. The compound of claim 30 further comprising at least one spacer sequence located between said effector segment containing said cysteinyl residue and an additional effector segment.

50. The compound of claim 49 wherein said spacer sequence comprises at least one (Ser₄Gly) or (Gly₄Ser) segment.

51. The compound of claim 50 comprising two (Ser₄Gly) or (Gly₄Ser) segments.

52. The compound of claim 1 comprising a single-chain binding polypeptide which is effective in binding two or more surface markers of a mammalian cell.
